(19) United States
(12) Patent Application Publication
(10) Pub. No.: US 2023/0346261 A1
(43) Pub. Date: Nov. 2, 2023

(54) SYSTEM AND METHOD FOR ANALYZING GAIT IN HUMANS

(71) Applicant: Walkbeat AB, Göteborg (SE)

(72) Inventors: Siddhartha KHANDELWAL, Göteborg (DE); Joao Elias BRASIL BENTES JUNIOR, Göteborg (SE)

(73) Assignee: Walkbeat AB, Göteborg (SE)

(21) Appl. No.: 18/245,986

(22) PCT Filed: Sep. 27, 2021
(86) PCT No.: PCT/SE2021/050941

§ 371 Date: Mar. 20, 2023

(30) Foreign Application Priority Data
Sep. 25, 2020 (SE) 2051119-2

(51) Int. Cl. A61B 5/11

(52) U.S. Cl. CPC A61B 5/112; A61B 2562/0219

(57) ABSTRACT

An analysis system (1) for assessing gait quality and/or gait-related health status of a human (5) is provided. The analysis In system (1) comprises at least a first and second sensor devices (20a, 20b) each arranged at one leg of a human (5). The at least first and second sensor devices (20a, 20b,) each comprise at least one 3-axis accelerometer (21) and at least one 3-axis gyroscope (22), and the sensor devices (20a, 20b) are configured to provide gait data (22a, 22b). The analysis system (1) comprises a computing unit (10) configured to receive said gait data (22a, 22b), analyze said received gait data (22a, 22b) from said at least first and second sensor devices (20a, 20b) for determining at least one gait parameters (210) related to stride characteristics of said human (5), and analyze the at least one gait parameter (210) to assess gait quality and/or gait-related health status of the human (5).

### TECHNICAL FIELD

The present invention generally relates to the field of analyzing the gait of a human, and more particularly to a system and method for analyzing gait.

### BACKGROUND

Gait analysis refers to a study of observing human locomotion provisioned by measuring instruments for measuring body movements and muscle activity. The measurements provided from measuring instruments in a gait analysis study may be used to assess and treat movement impairing conditions. Assessments include for instance classifying movement patterns to determine what and how well an activity is being performed. Low levels of physical activity have been associated with increased risk of chronic diseases and thus knowing which activities a person performs during a day gives insights into their overall health status. As such, numerous works have been dedicated to classifying daily-living activities using wearable sensors.

Over the years, studies have been dedicated to analyzing gait. These gait measurements relate to spatio-temporal measures such as speed, cadence or step frequency, stance time, swing time and double support time.

In light of the observations above, the present inventors have realized that there is room for improvements when it comes to technical provisions for analyzing gait and/or assessing gait quality.

### SUMMARY

It is accordingly an object of the present invention to mitigate, alleviate or eliminate at least some of the problems referred to above, by providing an analysis system for analysing gait quality of a human.

Other aspects of the invention and its embodiments are defined by the appended patent claims and are further explained in the detailed description section as well as on the drawings.

In a first aspect of the invention, an analysis system for assessing gait quality and/or gait-related health of an human is provided. The analysis system comprises at least a first sensor device arranged at a region of a first leg of the human, and a second sensor device arranged at a region of a second leg of the human, wherein the at least first and second sensor devices each comprise at least one 3-axis accelerometer and at least one 3-axis gyroscope, and wherein said at least first and second sensor devices are configured to provide gait data; and a computing unit configured to: receive said gait data from said at least first and second sensor devices, analyze said received gait data for determining at least one gait parameters related to stride characteristics of said human, wherein said at least one gait parameter comprises information of at least one computed energy density spectrum, and analyze the at least one gait parameter to assess gait quality and/or gait-related health status of said human.

In one embodiment, the at least one computed energy density spectrum comprises at least one energy density spectrum computed from both or either one of sets of acceleration signals or gyroscope signals included in said gait data.

In one embodiment, the computing unit is further configured to analyze the at least one energy density spectrum by: measuring the variability by comparing each energy density spectrum to itself over a predetermined time period, and/or measuring the symmetry by comparing an energy density spectrum of a left leg of the human to an energy density spectrum of a right leg of the human, and/or measuring the normality by comparing each energy density spectrum to at least one energy density spectrum of a leg from a reference population group exhibiting no gait pathology.

In one embodiment, the computing unit is configured compute accelerometer energy density spectrums by: receiving the sets of acceleration signals; for each set of received acceleration signals, computing a resultant acceleration signal; based on said computed resultant acceleration signals, determining if the human is performing a gait related activity or is inactive; and if it is determined that the human is performing a gait related activity, computing an accelerometer energy density spectrum for each resultant acceleration signal, wherein each accelerometer energy density spectrum corresponds to one leg of the human.

In one embodiment, determining if the human is performing a gait related activity further involves: computing a moving standard deviation signal of the resultant acceleration signals; generating a filtered acceleration signal by performing filtering of said computed moving standard deviation signal; and determining if a total number of elements of the filtered acceleration signal having a value greater than or equal to a value of a corresponding element of a predetermined walking threshold. The filtering may be performed using 1-D morphological filtering.

In one embodiment, wherein the computing unit is configured to compute gyroscope energy density spectrums by: receiving the sets of gyroscope signals; for each set of received gyroscope signals, computing a resultant gyroscope signal; and for each resultant gyroscope signal, computing a gyroscope energy density spectrum wherein each gyroscope energy density spectrum corresponds to one leg of the human.

In one embodiment, the computing unit is configured to combine the accelerometer energy density spectrum and the gyroscope energy density spectrum for assessing gait quality and/or gait-related health status of said human. In one embodiment, wherein the accelerometer energy density spectrum and/or the gyroscope energy density spectrum is used to measure fluctuations in gait over time.

In one embodiment, the computing unit is further configured to: receive at least one metadata associated with the human, analyze the at least two gait parameters and said at least one metadata to assess gait quality and/or gait-related health status of said human.

In one embodiment wherein the at least one metadata comprises one or more of: information of subject data of the human, information of person data of persons related or connected to the human, information of accessory data related to accessories of the human, and information of training data of the human. The metadata may include information relating to medications that the human is using.

In one embodiment, the at least one metadata is based on data received from at least one additional sensor and/or based on data being inputted to the system by a user. The at least one additional sensor may be one or more of: a GPS-sensor, a temperature sensor, a weather sensor, and a pulse sensor.

In one embodiment, the at least one gait parameters and the at least one metadata are analyzed by comparing them against one or more baselines and/or against historical data. In one embodiment, the computing unit is further configured to compute statistical data and/or historical data of the at least one metadata and the at least one gait parameter.

In one or more embodiments, the computing unit is further configured to store said assessed gait quality and/or gait-related health status and/or to communicate said assessed gait quality and/or gait-related health status to an external device having a display, wherein the external device is configured to present said assessed gait quality and/or gait-related health status to a user.

In one embodiment, the computing unit is further configured to generate and transmit a deviating signal to the external device if the at least one metadata and/or the at least two gait parameters exceeds a predetermined deviating threshold value.

In one embodiment, the assessed gait quality and/or gait-related health status is used to detect at least one of: one or more improvements in health status of the human, no or at least one minor change in the gait quality of the human, and/or an increase in risk of one or more injuries and/or diseases of the human.

In a second aspect of the invention, a method for assessing gait quality and/or gait-related health status of a human is provided. The human is being equipped with at least a first sensor device at a region of a first leg of the human and a second sensor device at a region of a second leg of the human, wherein the at least first and second sensor devices each comprise at least one 3-axis accelerometer and at least one 3-axis gyroscope, and wherein the at least first and second sensor devices are configured to provide gait data. The method involves: receiving said gait data from said at least first and second sensor devices; analyzing said received gait data for determining at least one gait parameter related to stride characteristics of said human, wherein said at least one gait parameter comprises information of at least one computed energy density spectrum; and analyzing the at least one gait parameters to assess gait quality and/or gait-related health status of the human.

The invention described herein has several benefits for medical professionals as well as the subject that is analysed. Moreover, the invention may provide benefits for personal trainers, physiotherapists, neurologists, caregivers R&D centers and universities. For the trainer the system allows to fine tune every aspect of training with gait insights to maximize performance and minimize risk of injury. The benefits will now be summarized herein.

One benefit includes that different performance parameters can be used to maximize gait quality and/or other measures depending on the situation, such as speed and endurance. Moreover, identification of which combination of training factors, e.g. surface/shoe/, etc. (metadata) may be provided, which leads to better performance and health over time. Additionally, the user may find and replicate gait signatures (humans), ultimately leading to better performance and healthier humans. Yet additionally, early detection of injuries and other health problems can be provided, and the tracking of rehabilitation processes for deciding when to resume training may be provided. Gait variability is the phenomenon of having changes in gait parameters from one stride to the next. Having a high gait variability is known to be common in individuals affected by neurodegenerative conditions such as Parkinson's disease and Huntington's disease in humans.

The system and method as claimed herein is furthermore beneficial for medical professionals. They can conduct fast gait tests with walking and running to detect even minor deviations, often in response to medications and/or interventions/treatments, which are difficult to catch with the naked eye. The medical professionals will have a tool to communicate with their patients using objective gait information as a basis during rehabilitation and recovery. The history of gait information can be used to improve future diagnosis. As such, benefits provided for the medical professionals may involve support in diagnosis based on current gait quality and history, development of injury/rehabilitation during follow-ups, and following, tracking and prescribing custom rehabilitation based on the patients initial response to medication, diagnosis or treatment. Moreover, benefits involve the sharing of objective analysis with the patient and the patients family for traceability and digital rehabilitation which can be used for future services.

The invention may also be beneficial for shoe makers, and shoe brands. They can conduct fast gait tests before and after wearing the specific shoe to make objective evaluation of shoeing quality and feet-shoe fit. The system and method as claimed herein give them a tool to fine tune the development of shoe process and technique to get the best performance from the user of the shoe. The history of gait information can be used to improve future shoe developments.

The system and method as claimed herein are also beneficial for researchers and R&D facilities in different fields. The system allows to collect precise, accurate movement data with time-synchronised inertial sensors that have global timestamps. The system will promote collaboration as well as conducting research on-the-go at remote locations with easy-to-manage database. As such, the benefits include, but are not limited to accessing data collections in remote locations, conducting extensive studies in the real world to open up new strains of research, information and learnings, and accessing all levels of information which ensures a wider sample size and generalization of research to all humans.

It should be emphasized that the term “comprises/comprising” when used in this specification is taken to specify the presence of stated features, integers, steps, or components, but does not preclude the presence or addition of one or more other features, integers, steps, components, or groups thereof. All terms used in the claims are to be interpreted according to their ordinary meaning in the technical field, unless explicitly defined otherwise herein. All references to “a/an/the [element, device, component, means, step, etc]” are to be interpreted openly as referring to at least one instance of the element, device, component, means, step, etc., unless explicitly stated otherwise. The steps of any method disclosed herein do not have to be performed in the exact order disclosed, unless explicitly stated.

### BRIEF DESCRIPTION OF THE DRAWINGS

Objects, features and advantages of embodiments of the invention will appear from the following detailed description, reference being made to the accompanying drawings, in which:

FIG. 1 is a schematic illustration of a non-limiting example of an analysis system in which embodiments of the present invention may be exercised.

FIG. 2 is a schematic illustration of a non-limiting example of an analysis system in which embodiments of the present invention may be exercised.

FIG. 3 is a schematic block diagram of a sensor device holder used for analysis in one embodiment.

FIGS. 4a-b are schematic block diagrams illustrating the basic internal hardware and software layout of a mobile communication terminal according to embodiments of the invention.

FIG. 5 is a schematic block diagram illustrating features forming part of assessing gait quality and gait-related health status according to embodiments of the invention.

FIG. 6 is a schematic block diagram illustrating features forming part of assessing gait quality and gait-related health status according to one embodiment.

FIGS. 7a-c are illustrations of gait parameters at least based on gait data from sensor devices according to embodiments of the invention.

FIGS. 8a-c are illustrations of gait parameters at least based on gait data from sensor devices according to embodiments of the invention.

FIGS. 9a-c are illustrations of gait parameters at least based on gait data from sensor devices according to embodiments of the invention.

FIGS. 10a-d are illustrations of different gait parameters from sensor devices according to embodiments of the invention.

FIGS. 11a-d are illustrations of different gait parameters according to embodiments of the invention.

FIGS. 12a-c are block diagrams illustrating procedural steps of assessing gait quality and gait-related health status using metadata and/or gait parameters according to embodiments of the invention.

FIG. 13 is a flowchart of a part of a method of assessing gait quality and gait-related health status according to embodiments of the invention.

FIGS. 14a-e are flowcharts of parts of a method of assessing gait quality and gait-related health status according to embodiments of the invention.

FIG. 15 is an illustration of an external device generally according to some embodiments of the invention.

FIG. 16 is a flowchart illustrating feedback loops when assessing gait quality according to one embodiment.

### DETAILED DESCRIPTION

Embodiments of the invention will now be described with reference to the accompanying drawings. The invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the particular embodiments illustrated in the accompanying drawings is not intended to be limiting of the invention. In the drawings, like numbers refer to like elements.

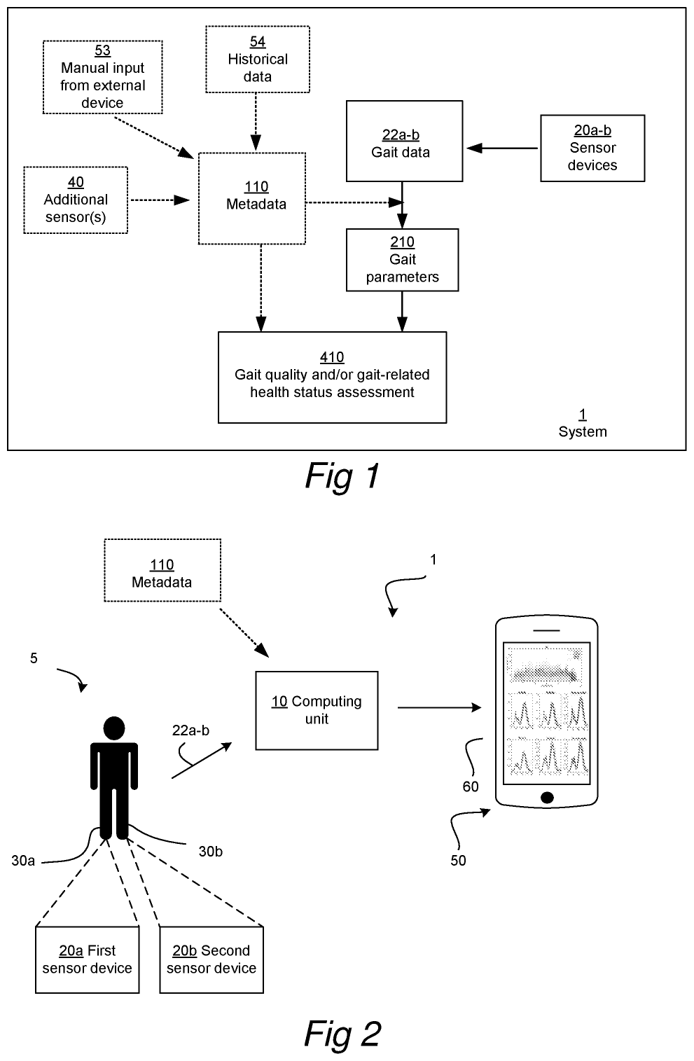

FIGS. 1 and 2 illustrate an analysis system 1 for assessing gait quality and/or gait-related health status generally according to an embodiment of the present invention.

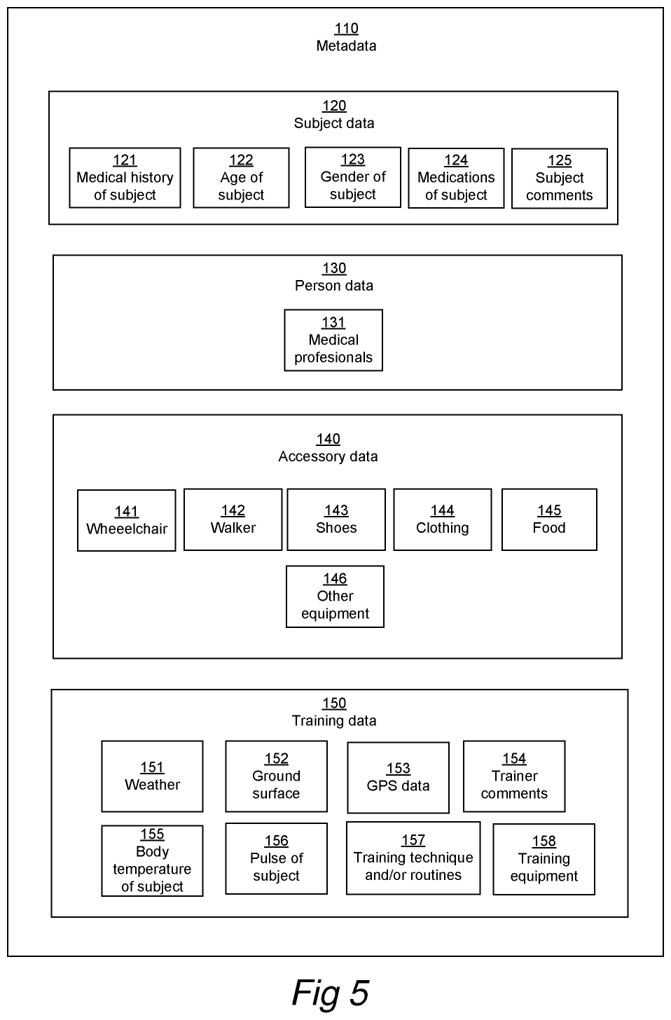

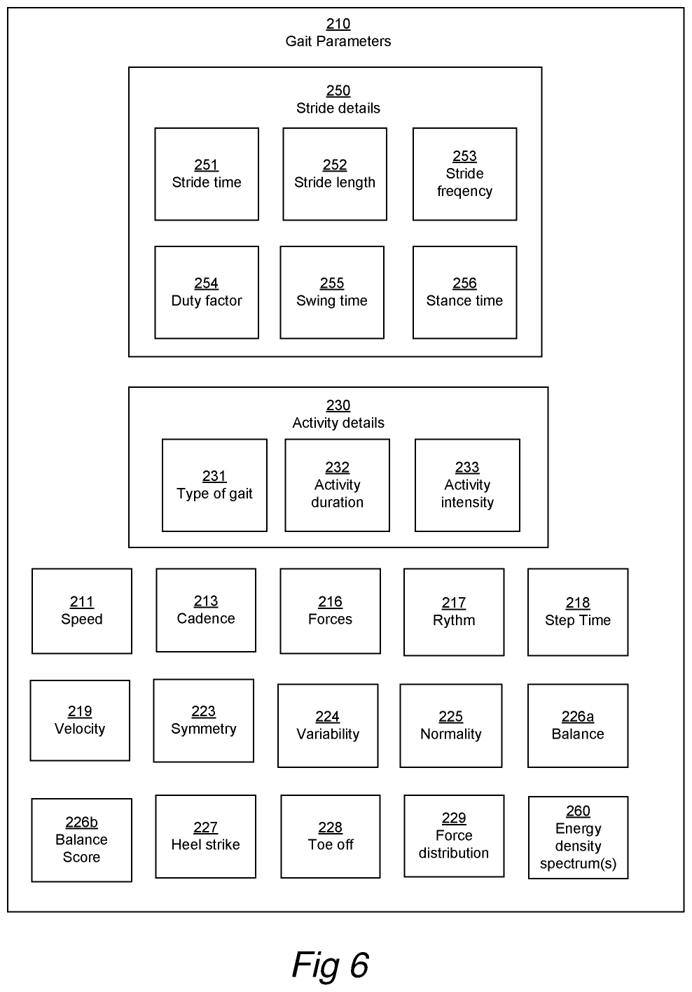

The system 1 comprises a plurality of sensor devices 20a-b that are configured to collect respective gait data 22a-b of the subject 5. The gait data 22a-b is evaluated and analyzed to generate gait parameters 210, which will be described more in detail with reference to FIG. 6, that are used to assess the quality of the gait. Metadata 110 relating to the subject 5 and its environmental factors, described more in detail with reference to FIG. 5, is collected manually by a user 53, automatically by the system 1 itself, historical data 54, and/or by one or more additional sensors 40. Additional sensors 40 could be a pulse sensor, a temperature sensor, a weather sensor and/or a GPS.

The system 1 comprises one or more subjects 5 being subjects for gait analysis. In the exemplifying embodiment as illustrated by FIG. 1b, a human 5 is being analyzed. The information described throughout the present disclosure will be directed at humans 5. The terms “subject” and “human” will be used interchangeably throughout this disclosure, but are both referring to the same subject, i.e. the human 5 (such as the one shown in FIG. 2).

The analysis system 1 for assessing gait quality and/or gait-related health status further comprises at least a first and a second gait sensor device 20a, 20b configured to provide respective gait data 22a, 22b of the human 5. In yet other embodiments, the system 1 may comprise an arbitrary number of sensor devices 20a-b positioned on different body parts and configured to store and retrieve gait data 22a-b. In one embodiment, although not shown, the system 1 comprises four sensors, where two sensors are attached to the leg of the subject 5 and two sensors are attached to the arms or wrists of the subject 5. The sensors 20a-b described herein may be identical to the sensors arranged at the arms or wrist of the subject 5.

Throughout the present disclosure, it is described that gait data 22a-b is received from a respective sensor device 20a-b. This is referring to that each sensor device 20 is configured to provide one or more bits or streams of gait data 22a-b, for a respective leg 30a-b, of the human 5.

Each sensor device 20 may be arranged at a location suitable for providing accurate gait data 22a-b of the subject 5. For instance, the sensor devices 20 may be arranged at the legs 30a-b of the human 5. More specifically, the sensor devices may be arranged just above the ankles at each leg 30a-b.

As seen in FIGS. 1-2, the gait analysis system 1 further comprises a computing unit 10. The computing unit 10 may be a cloud-computing unit 10 being included in a distributed cloud network widely and publicly available, or limited to an enterprise cloud. For instance, cloud-computing technologies include, but are not limited to Amazon EC2, Google App Engine, Firebase or Apple iCloud. The computing unit 10 is at least configured to receive gait data 22a-b from the sensor devices 20a-b. Further, the computing unit 10 is configured to analyze said received gait data 22a-b for assessing gait quality relating to the gait characteristics of the human 5. In a preferred embodiment, received gait data 22a-b is analyzed to assess gait quality, wherein the gait quality is related to stride characteristics of a human 5. Moreover, the computing unit 10 may further be configured to receive and analyze metadata 110. The computing unit 10 may further be configured to perform a gait quality and/or gait-related health status assessment 410 based on the metadata 110 and the gait parameters 210.

Additionally, the computing unit 10 may also be configured to store the metadata 110 and gait history for long-term analysis.

The computing unit 10 is further configured to communicate the assessed gait quality and/or gait-related health status assessment 410 to an external device 50. The external device 50 may be embodied as a mobile terminal, for instance a mobile phone, laptop computer, stationary computer or a tablet computer. Preferably, the external device 50 has a display 60. The display 60 may be a touch screen display or a non-touch screen. The display 60 is configured to present information of the analysis performed by the computing unit 10 and/or the analysis performed by the external device 50. Preferably, the external device 50 is configured to present the assessed gait quality and/or gait-related health status. As will be discussed more in detail later on, this information may be presented as different graphs and/or different values (such as a score, index value, etc.). It should be noted that the analysis performed in the computing unit 10 also could instead be partly or fully performed in the external device 50.

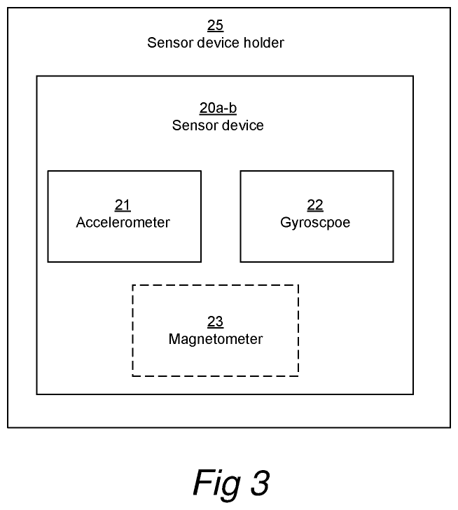

As illustrated in FIG. 3, the gait sensor devices 20a-b may be attached to a subject using a sensor device holder 25. The sensor device holder 25 may be any attachment means such as for example an adhesive material or a strap, belt, harness, band or similar. In one embodiment, the gait sensors device 20 is arranged in a sensor device holder 25 that is clipped on to the socks (not shown).

FIG. 3 further illustrates an exemplified embodiment of a sensor device 20a-b. Each sensor device 20a-b preferably comprises at least one accelerometer 21, and at least one gyroscope 22. In a preferred embodiment, the sensor device 20a-b further comprises at least one magnetometer 23. In one embodiment, each sensor device 20a-b comprises at least one 3-axis accelerometer 21, at least one 3-axis gyroscope 22 and at least one magnetometer 23 configured to provide gait data 22a-b. As will be described more in detail with reference to FIG. 5, gait data 22a-b may be locally stored, retrieved continuously or at a predetermined timely basis. In one embodiment the provided gait data 22a-b include sets of acceleration signals aₓ, a_{y}, a_{z} retrieved from at least one 3-axis accelerometer 21, sets of gyroscope signals gₓ, g_{y}, g_{z} retrieved from at least one 3-axis gyroscope 22 and sets of magnetometer signals retrieved from at least one magnetometer 23. Accordingly, the sets of acceleration, gyroscope, and magnetometer signals may be included in the gait data 22a-b.

The magnetometer 23 measures the magnetic field or magnetic dipole moment. The magnetometer 23 may measure the direction, strength and/or relative change of a magnetic field at a particular location. In one embodiment the magnetometer 23 is a vector magnetometer 23 that can measure one or more components of the magnetic field electronically. In one embodiment, the magnetometer 23 is a scalar magnetometer that measures the total strength of the magnetic field to which it is subjected, and not its direction. In one embodiment, the magnetometer 23 is used in conjunction with a 3-axis accelerometer to produce orientation independent accurate compass heading information. In one embodiment, the magnetometer 23 is a 3-axis magnetometer.

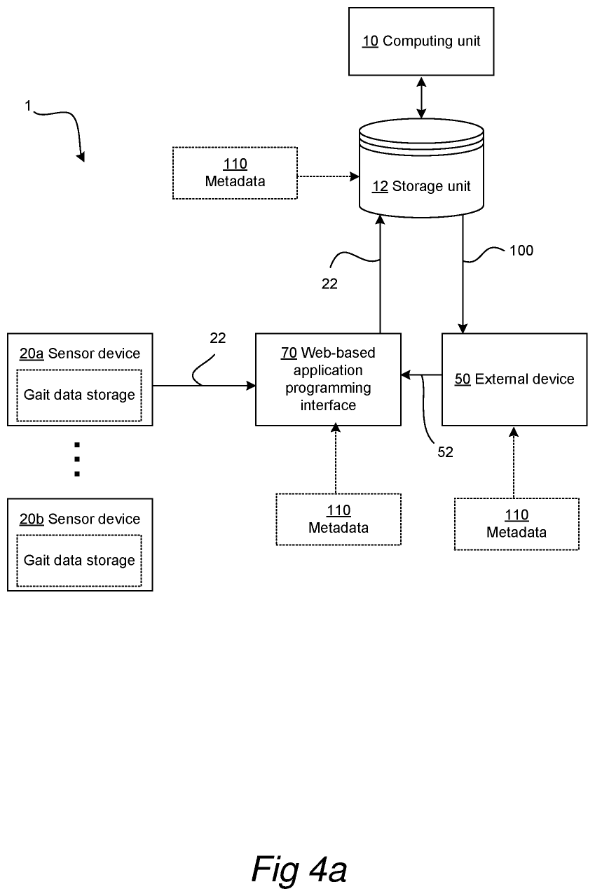

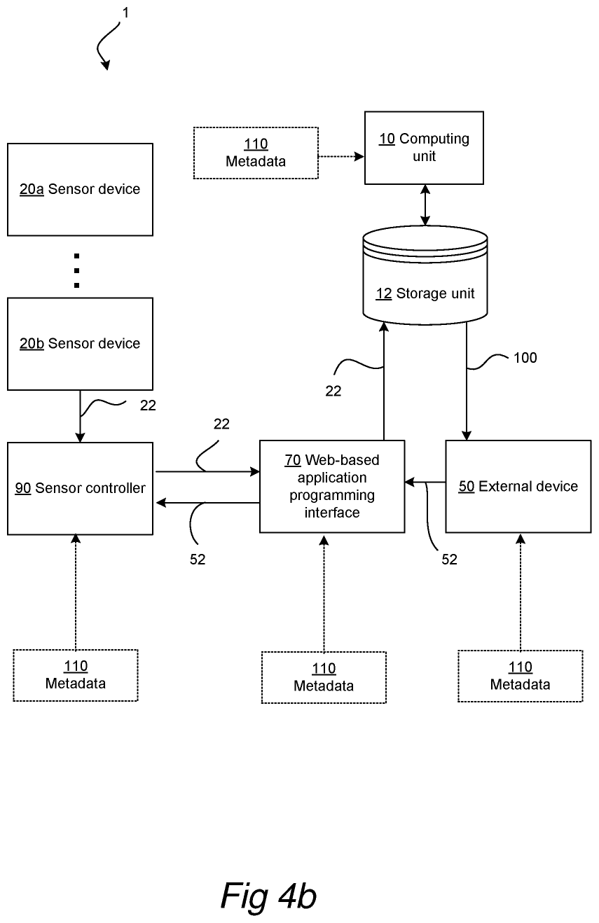

FIG. 4a illustrates a schematic block diagram with the basic internal hardware and software layout of an analysis system 1 according to one embodiment. In addition to the elements described with reference to FIG. 1b, the system 1 may further comprise a storage unit 12 and a web-based API (Application Programming Interface) 70. The web-based API 70 is configured to receive an event request 52 from the external device 50, instructing the web-based API 70 to initiate a gait analysis event. The web-based API 70 is further configured to activate the sensor devices 20 for providing gait data 22 to the web-based API 70 using short-range communication technologies. Examples of such technologies are short-range standards IEEE 802.11, IEEE 802.15, ZigBee, WirelessHART, WIFI and Bluetooth® to name a few. It should be noted that, as is commonly known, the web-based API is arranged to communicate according to more than one technology and many different combinations exist. Further, peer-to-peer connection between the web-based API 70 and the external device 50 may be established using protocol standards such as for instance HTTP, HTTPS, WebRTC, QUIC, IPFS.

Moreover, communications may also be based on transferring data via IoT-services (Internet of Things). In different embodiments of the invention, different IoT-protocols may be utilized. For instance, protocols include, but are not limited to Bluetooth®, WiFi, ZigBee, MQTT IoT, CoAP, DDS, NFC, AMQP, LoRaWAN, RFID, Z-Wave, Sigfox, Thread, EnOcean, celluarly based communication protocols, or any combination thereof.

The storage unit 12 may be run on a cloud-computing platform, and connection may be established using DBaaS (Database-as-a-service). For instance, the storage unit 12 may be deployed as a SQL data model such as My SQL, PostgreSQL or Oracle RDBMS. Alternatively, deployments based on NoSQL data models such as MongoDB, Hadoop or Apache Cassandra may be used. DBaaS technologies include, but are not limited to Amazon Aurora, EnterpriseDB, Oracle Database Cloud Service or Google Cloud. Preferably, the storage unit 12 is deployed on the same platform as the computing unit 10 deployment.

As indicated in dashed lines, gait data 22a-b may be stored locally in the sensor units 20a-b. The gait data 22a-b may be stored locally in the sensor units before being transmitted to a web-based application programming interface 70 or being directly transmitted to the storage unit 12. The computing unit 10 then computes gait parameters 210.

Metadata 110 could be received to the system 1 by either the web-based application programming interface 70, the external device 50, or by the computing unit 10.

In FIG. 4b, a schematic block diagram illustrating another embodiment of the invention is presented. Herein, the gait analysis system 1 further comprises a sensor controller 90 configured to receive the event request 52 from the external device 50 via the web-based API. The sensor controller 90 further comprises an activator application which is configured to control the activation of the sensor devices 20. The activation may be performed automatically as a response to having received an event request 52. Alternatively, the activation may be performed manually by a user. Similar to the embodiment discussed when referencing FIG. 4a, the sensor controller 90 comprises a communication interface based on any short-range communication technology as mentioned. The activator application may for instance be embodied as a mobile application or a web-based application, configured to respond to user input using e.g. physical buttons, touch screen functionalities, audible input, sensorial input, or any combination thereof.

As shown in FIG. 4b, some kinds of metadata 110 that are originating from at least one additional sensor 40, can be stored and processed in the sensor controller 90. This may for example be the case with metadata such as weather data, GPS data, pulse data or temperature data. Although not shown, the system 1 may further comprise means for providing secure communication between software and hardware components of the system. In order to ensure secure communication, messages may be encrypted, encoded, enciphered using a variety of cryptographic hash functions. For instance, SHA-1, SHA-2, CRC32, MD5, or any other commonly used hash function may be used.

The following is an example embodiment of the process of generating a health and performance assessment in an analysis system 1 generally according to the present invention shown in FIG. 4b. The required information to start a new analysis is initiated by the user of the external device 50. The user may activate a trial event by requesting an event request 52 to the web-based API. This may be done using for instance a phone app, a tablet app, a web service or similar, installed on the external device 50. Consequently, the web-based API receives the event request 52, and routes it to the sensor controller 90. The sensor controller 90 triggers a sensor activation signal, which may be communicated via a short-range communication standard. The communication may also be performed using an IoT-service as discussed above. In response to the activation signal from the sensor controller 90, each sensor device 20a-d is configured to stream gait data 22a-b in the form of acceleration data, gyroscope data and magnetometer data to the sensor controller 90 via short-range communication standards. The data may also be transmitted via IoT-services.

When the sensor controller 90 has received a set of acceleration data, gyroscope data and magnetometer data corresponding to a predetermined quantity, the web-based API 70 receives the sets from the sensor controller 90. For instance, the sensor controller 90 may receive approximately 10 seconds of raw data retrieved by the sensor devices 20a-b. If the sensor devices 20a-b are configured to a sampling frequency in hertz, e.g. 128 hertz, the sensor controller 90 may receive approximately 1300 data points of raw data. The web-based API 70 is configured to transmit the retrieved sets of acceleration data, gyroscope data and magnetometer data to the storage unit 12 using for instance a DBaaS-technology as described above.

Subsequently, the computing unit 10 reads the data from the storage unit 12, performs the gait analysis to generate gait parameters 210. The gait parameters 210 are used alone or together with metadata 110 in order to gain a quality and health assessment. This assessment may be transmitted back to the storage unit 12 which stores the received analysis and transmits it to the external device 50.

Attention is now directed towards FIGS. 5 to 16. Herein, analysis system properties and methods are provided for assessing the gait quality and/or gait-related health status of the human 5.

Before turning into details of how the data is computed, evaluated and used, the details of the terminology metadata 110 and gait parameters 210 will be described with reference to FIGS. 5 and 6. Both the metadata 110 and the gait parameters 210 affect the gait quality and/or gait-related health status of the human 5.

A schematic illustration of the details of metadata 110 are illustrated in FIG. 5. The metadata 110 should be seen as parameters that can be categorized into different categories. In the example illustrated in FIG. 5 the metadata 110 is categorized into four different categories, but the present disclosure is not limited to the categories illustrated in FIG. 5. Further categories of metadata 110 than the categories illustrated in FIG. 5 can exist. In the exemplary embodiment the data is presented for a human.

In FIG. 5, a first category of metadata 110 is related to subject data 120, a second category is related to person data 130, a third category is related accessory data 140 and a fourth category is related to training data 150. These categories will now be described in more detail. The first category related to subject data 120 comprises information relating to the subject (such as the subject (human) 5) medical history 121, age of the subject 122, gender of the subject 123, comments from the subject 125, and/or information relating to medications 124. The information relating to medications 124 may comprise information such as timings of the medications, the amount/quantity/dosage of the medication, combination of different medications and so on. The comments from the subject 125 may comprise dairy data, such as a patient health dairy. Metadata 110 in the category subject data 120 is not limited to the subject data 120 listed, other types of subject data 120 can also exist in this category.

In the embodiment where the subject is a human 5, the category medical history 121 may comprise previous or current diseases, or DNA-data and other information relating to the medical history of one or more relatives of the subject 5, and so forth. The category age 122 comprises information about the age of the subject 5, such as the number of years and/or months. The category gender 123 preferably comprises information about the gender and/or sex of the subject 5. Hence, the gender 123 category may include information if the subject 5 is a male or female. The category medications of subject 124 preferably comprises information relating to formerly and/or currently administered medications 124, and/or the quantity of said administered medications at the time of administration. Moreover, medications 124 may also comprise information relating to what specific type of medication 124 was administered, for what purpose, the producer thereof, the batch number of the administered medication 124, and so forth.

The second category of metadata 110 relates to person data 130. In the category person data 130 there are metadata 110 such as medical professionals 131 associated with the subject 5. Medical professionals 131 may include persons involved in any type of profession in the field of medicine, including but not limited to doctors, nurses, caretakers, rehabilitation professionals, masseurs, or practically any type of people that are or formerly have been related to the human 5 in some way such that it has or is affecting the gait quality and/or gait-related health status of the human 5.

The third category related to accessory data 140 may for instance comprise information relating to one or more accessory/accessories that the human 5 may use. Such accessories may for example be one or more of a wheelchair 141, walker 142, shoe 143, clothing 144, food 145, or other equipment 146. The food 145 may include type of food (such as brand and/or ingredients) and/or the amount of food. The information may further include the time for each delivery of food (such as morning, before training, etc.). Metadata 110 in the category accessory data 140 is not limited to the accessory data 140 listed, other types of accessory data 140 can also exist in this category.

A fourth category is related to training data 150 and comprise information relating to a training session. The training data may for example be one or more of weather 151, ground surface 152, GPS data 153, body temperature 155 of the human 5, pulse 156 of the human 5, training techniques and routine 157, trainer comments 154, training equipment 158 as well as other training related parameters. Training data 150 may comprise information regarding training method, training regime, training style, training knowledge and training routine. The weather data 151 may contain information regarding temperature, wind, sun, clouds and so on. The weather data 151 may be collected from a cloud information system originating from weather stations or be gathered from weather sensors. The GPS data 153 may be collected from one or more additional sensors 40, such as a GPS-sensor. The information relating body temperature 124 and/or pulse 125 may be received from one or more additional sensors 40. The additional sensors 40 may for example be temperature sensors, pulse sensors or health sensors configured to measure temperature and/or pulse. The metadata 110 originating from an additional sensor 40 may be referred to as sensor based metadata 110. Hence, weather, GPS, pulse and/or body temperature may be seen as sensor based metadata 110. Training equipment 158 may comprise information relating to different types of equipment used in a training session. Training equipment 158 may include different types of treadmills with information regarding e.g. different inclinations. Training equipment 158 may also include oxygen masks commonly used for VO2Max tests, and the oxygen mask can be associated with the pulse 156 of the subject 5, for instance.

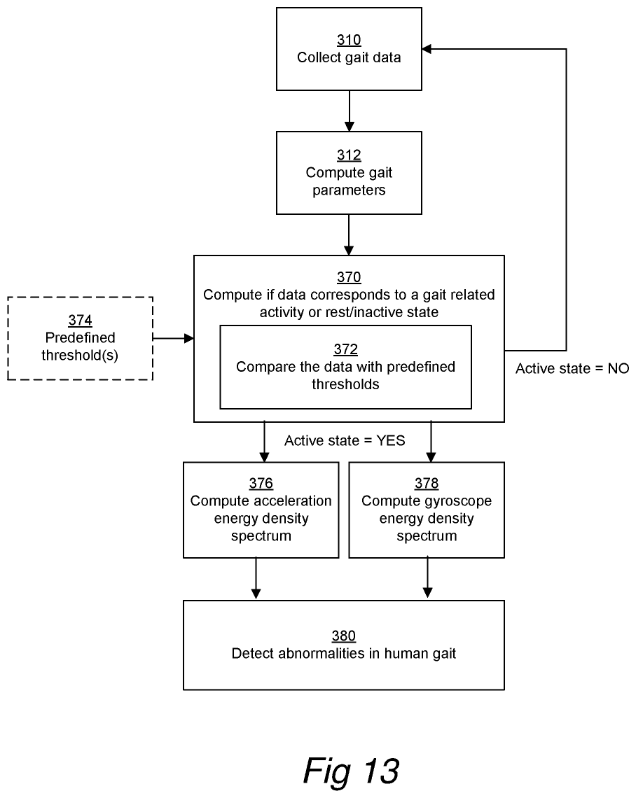

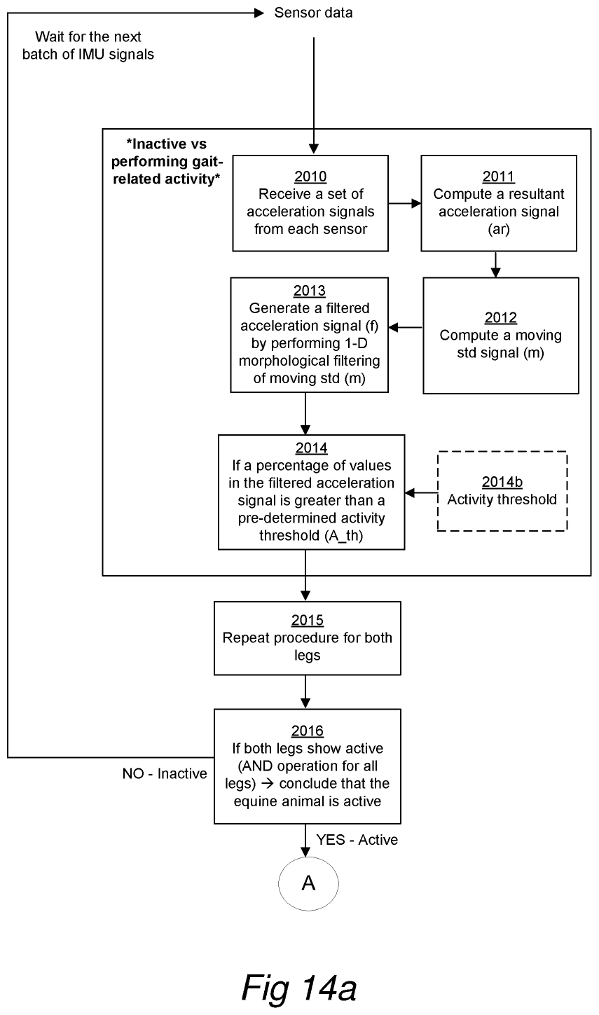

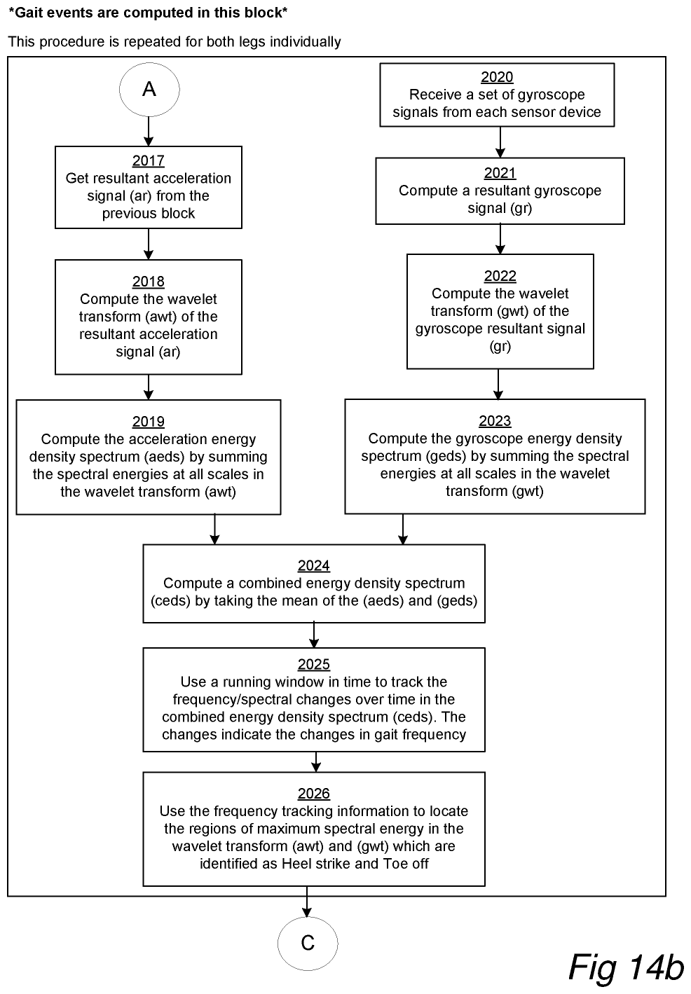

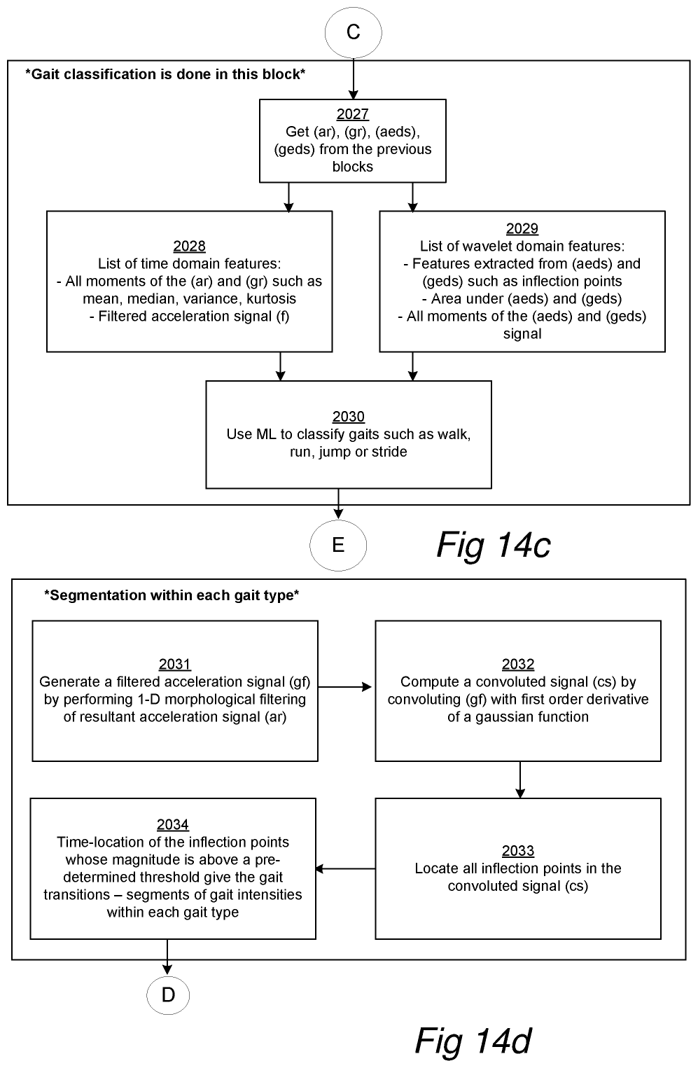

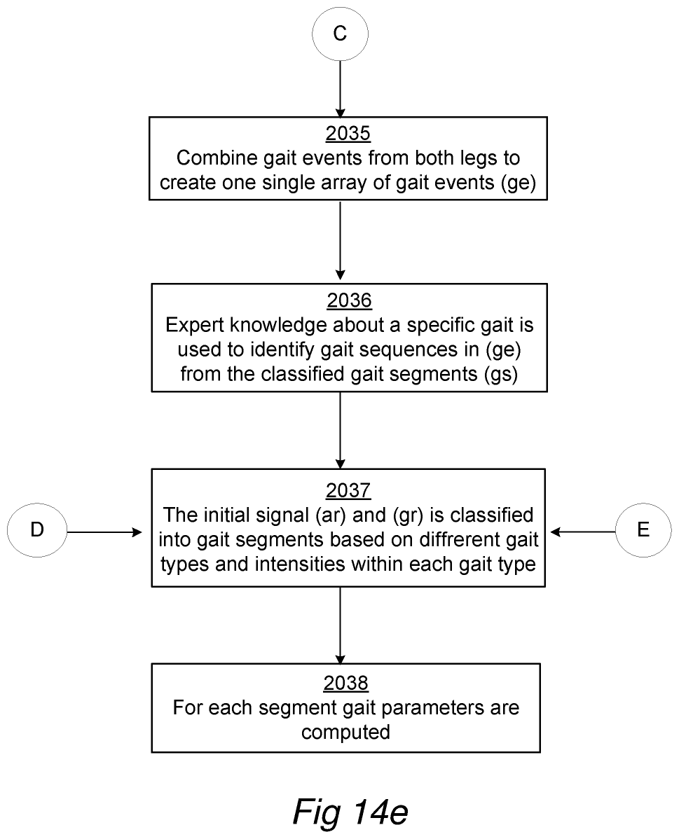

Now turning to FIG. 6 illustrating a block diagram of gait parameters 210 that are determined at least based on the gait data 22a-b received from the gait sensor devices 20a-b according to an exemplary embodiment of the present disclosure. In the example illustrated in FIG. 6 the gait parameters 210 are categorized into different categories, but the present disclosure is not limited to the categories illustrated in FIG. 6. Further categories of gait parameters 210 than the categories illustrated in FIG. 6 can exist. Gait parameters 210 are related to stride characteristics of the human 5. Stride characteristics may comprise any type of information associated with the human locomotion, i.e. a pattern of limb movements. Information associated with the human locomotion may, for instance, be retrieved as the gait data 22a-b by the at least first and second sensor devices 20a-b and further analyzed by the computing unit 10. As such, the stride characteristics may comprise one or more energy density spectrums computed from the gait data 22a-b. This will be thoroughly discussed later on with reference to FIGS. 13 and 14a-e.

The gait parameters 210 may comprise information relating to activity details 230. Activity details 230 may comprise information such as type of gait 231, activity duration 232 and/or activity intensity 233. The type of gait 231 may for a human be walking, running, jumping or striding. The activity duration 232, or training time, is the time which the activity lasts, for example measured in seconds or minutes. The activity intensity 233 may be measure as “low”, “medium” and “high” and the definition may be based on stride details 250, speed 211 and/or forces 216.

The gait parameters 210 may comprise information relating to stride details 250. Stride details 250 may for example comprise information about stride time 251, stride length 252, stride frequency 253, duty factor 254, swing time 255 and/or stance time 256.

Stride time 251 is the time between two consecutive heel strikes by the same leg, also known as one complete gait cycle. This is usually expressed in seconds. Stride length 252 is the distance covered between two consecutive heel strikes or toe-offs. This is either measured directly or is computed as the equal to the product of stride time and speed. The stride length is usually expressed in foot or meters. Stride frequency 253 is the number of strides taken in a given time, this is usually expressed as strides per second or Hz.

The duty factor 254 is the ratio of stance time and stride time. The duty factor is expressed as either a fraction between 0 and 1 or as a percentage between 0% and 100%. The swing time 255 is the time a foot/leg is in the air/not in contact with the ground during one complete gait cycle. This is usually expressed in seconds. The stance time 256 is the time a foot/leg is in contact with the ground during one complete gait cycle. This is usually expressed in seconds.

The gait parameters 210 could also be one or more of the following: speed 211, step time 218, cadence 213, velocity 219, forces 216, force distribution 229, offsets 222, rhythm 217, heel strike 227, toe off 228, balance 226a, balance score 226b, symmetry 223, variability 224 and normality 225. The gait parameters 210 further include one or more energy density spectrums 260.

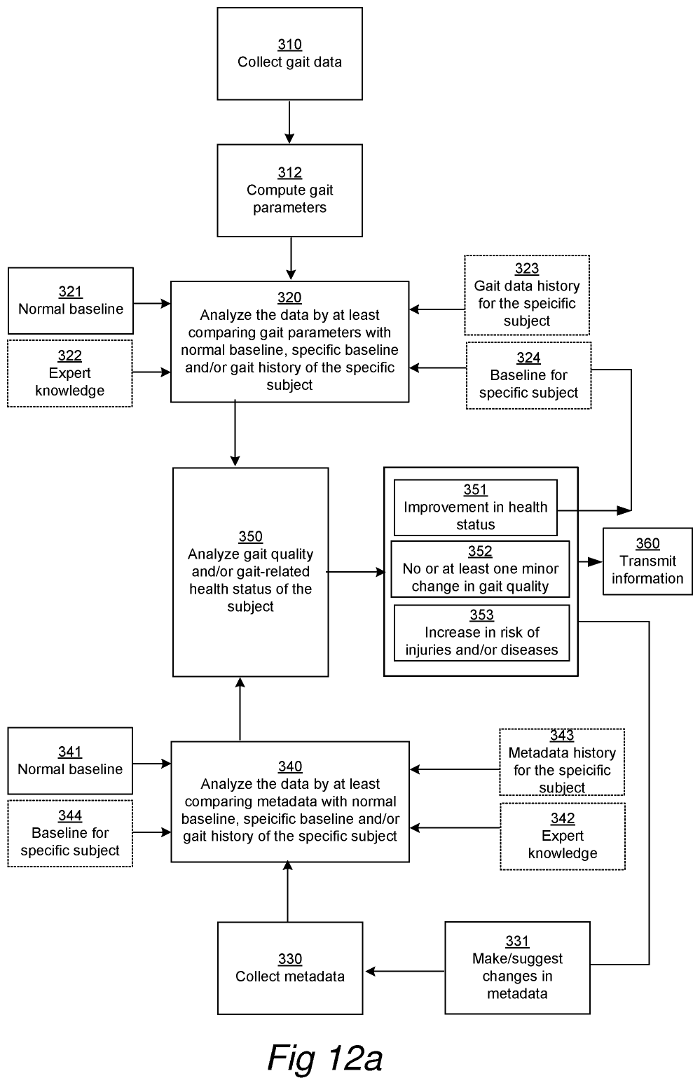

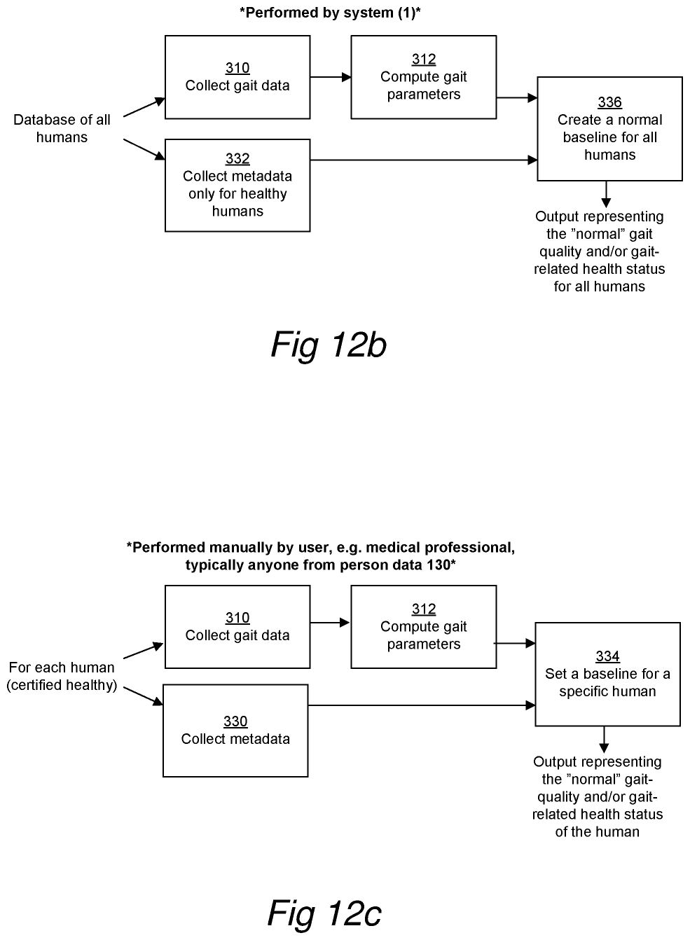

The energy density spectrums 260 are calculated based on the retrieved gait data 22a-b. Energy density spectrums 260 are used for analysing gait quality as they reveal any fluctuations in gait. Hence, the energy density spectrum(s) 260 may provide information relating to variation in gait. The energy density spectrums 260 may be assessed to detect 380 gait abnormalities. The energy density spectrums 260 and calculations thereof will be discussed thoroughly later on with reference to FIGS. 12-14.

The step time 218 is the time between two consecutive heel strikes, expressed usually in seconds. The cadence 213 is number of steps taken in a given time, usually steps per minute. The speed 211 is distance covered by the center of mass of the human in a given time. The speed 211 is either measured directly or computed as the equal to the product of stride length and stride frequency. The speed 211 is usually expressed as km/hr or m/s. The velocity 219 is speed 211 with a heading or specified direction. Force within gait cycle 229 is force experienced by the sensor positioned at a region of each leg during different phases of one complete gait cycle, such as heel strike, stance, mid-stance, toe-off, swing, mid-swing. This is usually expressed in Newton or g.

The gait rhythm 217 is the uniformity and consistency of the time elapsed between the Heel-Strikes or Toe-Offs of consecutive steps. The rhythm 217 is measured as the ratio of the time elapsed between Heel-strikes or Toe-offs of consecutive steps. As such rhythm 217, is expressed as a number between 0 and 1 or as a percentage between 0% and 100%, over time.

The heel strike 227 is the moment when the heel (full or in part) makes contact with the ground. Toe off 228 is the moment when the toe leaves contact with the ground. Variations may be realized for humans 5 that walk without contacting the ground with their heels, for instance only with a part of their fore foot and/or fore foot and middle foot. For instance some running techniques do not necessarily rely on contacting the ground with the heel.

Symmetry 223 is the ratios of parameters that compare left and right side of the body. Another example is ratio of stride times during normal walking, pacing or running. Variability 224 is the deviation of parameters for each leg 30a-b or the human 5 as a whole, when compared to themselves, over time. Hence, gait variability 224 is the phenomenon of having changes in gait parameters 210 from one stride to the next. Normality 225 is the deviation of parameters for each leg 30a-b or the human 5 as a whole, when compared to a normal population, over time.

Balance 226a is the overall force profile that takes into account the differences in the left and right side of the body. A Balance score 226b can also be computed by measuring the deviation from a normal gait rhythm 217.

The gait parameters 210 may be assessed based on its average value, as well as on its minimum and maximum value. The gait parameters 210 may be used alone or together when analysing the gait quality and thus also the gait-related health status of the human 5.

Some of the gait parameters 210 are assessed using one or more metadata 110. In one embodiment, some of the gait parameters 210 are assessed using sensor based metadata 110, such as for example a GPS-signal. In other embodiments, the gait parameters 210 are based solely on the gait data 22a-b provided by the sensors 20a-b. In yet one embodiment, the gait parameters 210 are based on the gait data 22a-b together with metadata 110 that is inputted by a user. In one embodiment, the gait data is used together with GPS-data in order to gain more accurate information relating to gait parameters regarding position and velocity. However, it should be noted that no GPS-signal, or other sensor based metadata 110, is essential in order to determine gait parameters 210.

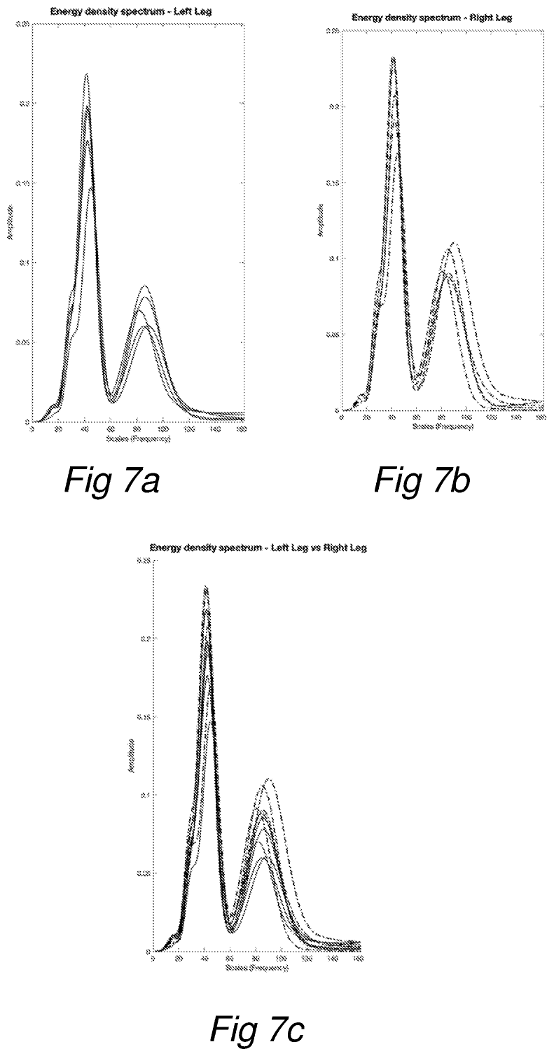

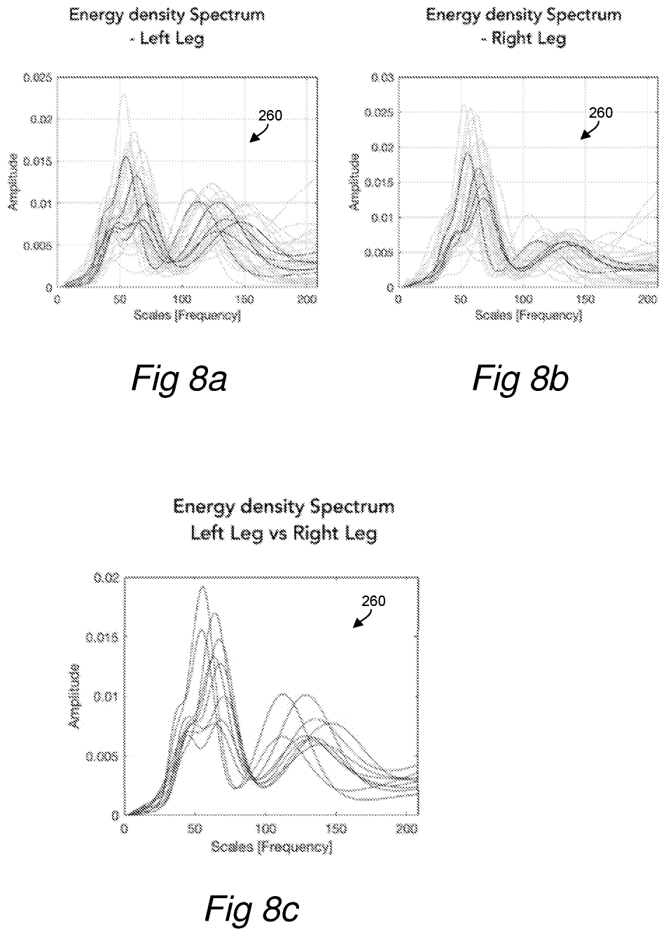

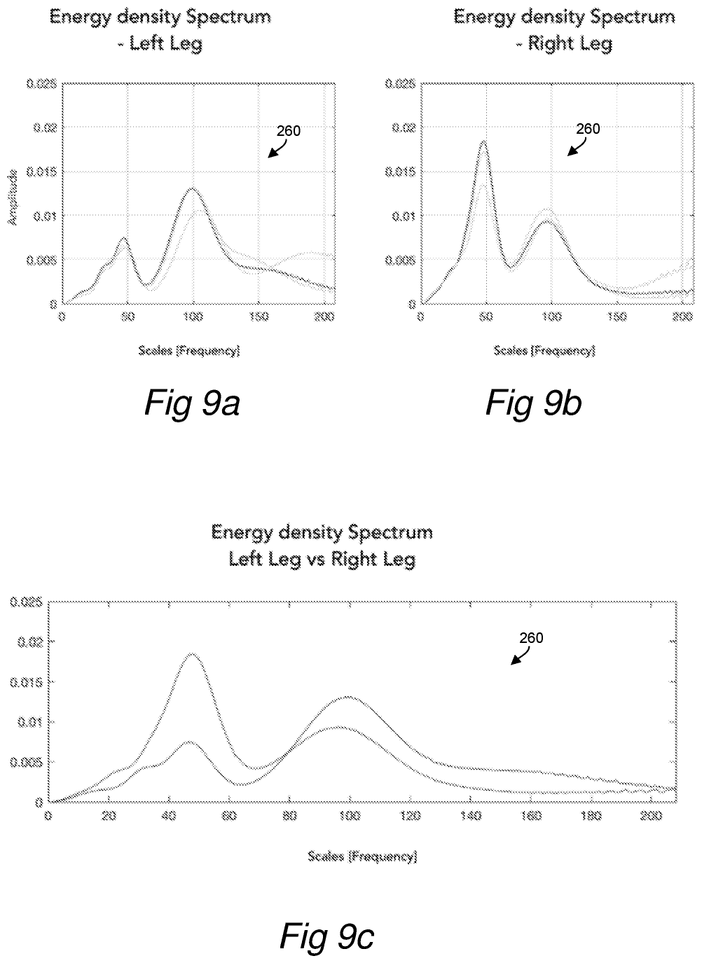

FIG. 7-9 illustrates energy density spectrums 260 for healthy and non-healthy subjects 5. FIG. 7 shows a healthy human 5, whereas the data in FIG. 8-9 indicates that the human 5 is unhealthy in some way. The data in FIGS. 8-9 may for example indicate that the human 5 is suffering from a disease, such as for example from Parkinson's disease, Stroke, a orthopedic injury such as for example an ankle injury. The signals may accordingly show early signs that a human 5 is suffering from Parkinson's disease. Other types of patterns may be indicative of other types of diseases. The subject matter as disclosed in the present application is not limited to identification of one particular type of disease, as the technical provisions may preferably serve as means for identifying practically any type of disease which can have an effect on a human's 5 gait quality and/or gait-related health status.

FIG. 7a shows a computed energy density spectrum 260 for a left leg, and FIG. 7b shows a computed energy density spectrum 260 for a right leg. In FIG. 7c, the energy density spectrum for both legs are combined in one illustration. The same is true for FIG. 8a-c and FIG. 9a-c, although with different data sets. The energy density spectrums can be evaluated further by investigating symmetry, normality and variability. Using these analysis, gait abnormalities can be early detected, mitigated, etc. by observing the energy spectrums over time. In one embodiment, features from energy density spectrums 260 can be extracted to classify pathologies, early detect disease/injury, etc.

As disclosed in the subject matter above related to the resultant acceleration signal aᵣ, the gyroscope acceleration signal gᵣ, and potentially also the magnetometer acceleration signal, can be assessed to identify other or similar type of gait-related diseases in humans 5.

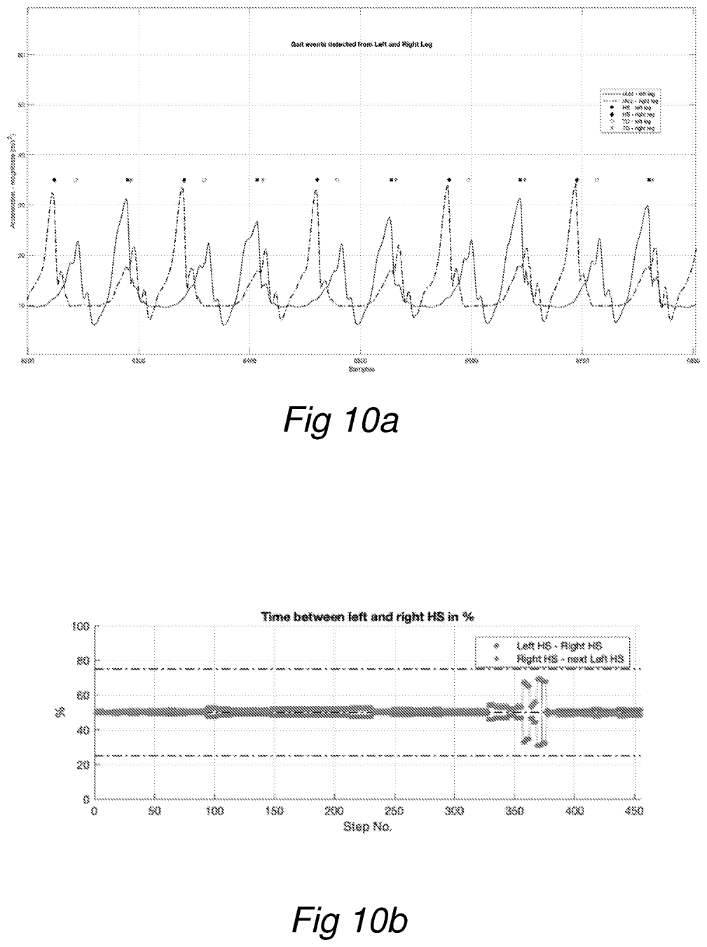

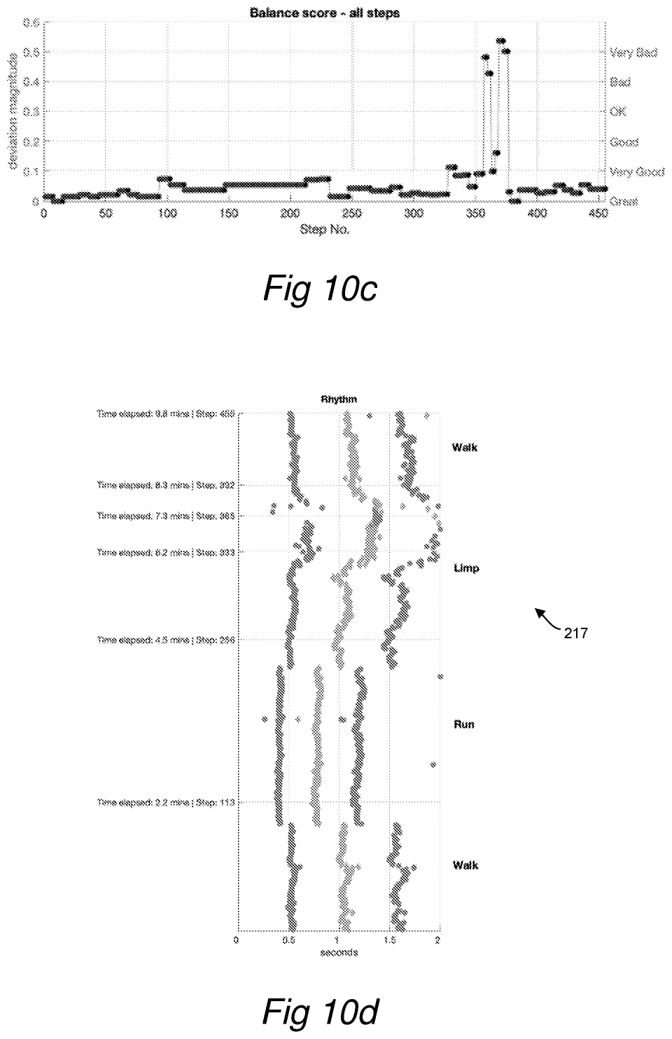

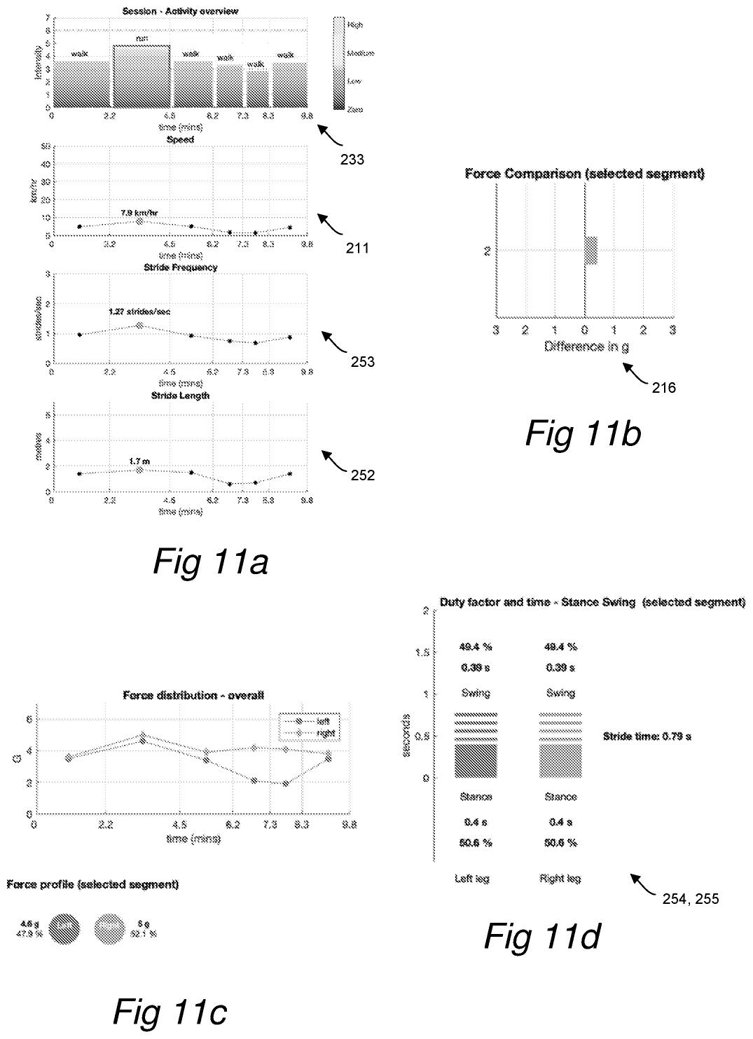

Some of the gait parameters 210 discussed in relation with FIG. 6 are illustrated in FIGS. 10-11. The exemplary embodiments shown in FIGS. 10-11 are data collected from a human 5.

FIG. 10a illustrates computed gait events that are detected from the left and right leg. In FIG. 10a, data is received from a 3-axis accelerometer 21, i.e. a set of accelerometer signals. The illustration shows the resultant acceleration signal ar computed from one or more sets of acceleration signals ax, ay, az received from the sensors 20a-b according to some embodiments of the invention. The data shown in FIG. 10a corresponds to accelerometer data associated with the gait of a walking human 5 that has a healthy gait status/quality. It also shows gait parameters 210 computed from the gait data 22a-b as received from the sensors 20a-b according to some embodiments of the invention. As shown, the gait parameters of Heel Strike (HS) 227 and Toe off (TO) 228 detected using the algorithm explained in FIG. 14b.

As seen in FIG. 10a, the two signals are occurring at a recurring frequency and amplitude for a long time period. This is an indication of a healthy subject 5. If the human 5 is having a limping gait quality, i.e. non-healthy, the two signals would not occur at a recurring frequency and/or amplitude during the same time period as the human 5 indicated in FIG. 10a.

FIG. 10b shows another gait parameter which illustrates the time expressed as a percentage between a left and right heel strike. The illustrations show the % over number of steps.

FIG. 10c shows another gait parameter, which is the balance score. In this illustration, the balance score is shown for all steps, and illustrates the deviation in magnitude from gait rhythm 217, over number of steps. This can for example be graded as Great, Very good, good, OK, bad and very bad.

FIG. 10d illustrates the gait rhythm 217 of a human 5 that is walking, running and limping. The analysis may include analysing the magnitude of frequency/rhythm over time for the different legs either alone or in combination. In this way it is possible to determine that the human 5 is limping or if it has a normal gait rhythm 217 and speed 211. The analysis may further include analysing the cadence (strides/minute) at a specific step as well as over time.

FIG. 11a shows an activity overview of one training session. In the top illustration the activity intensity 233 of the training session is shown over time, and the intensity levels are classified as zero, low, medium and high. In the second top illustration, the speed 211 is illustrated over time. The third top illustration shows the stride frequency 253, shows as strides/second over time. The lowest illustration in FIG. 11a shows the stride length 252, measures in metres over time.

FIG. 11b shows a force comparison 216 for a selected segment. FIG. 11c shows the overall force distribution as well as the fore profile for a selected segment.

FIG. 11d illustrates a duty factor 254 as well as the stance time 256 and swing time 255 for a selected segment. This is illustrated for each leg of the human 5. For example, for the right leg the swing time is 0.39 s and the stance time is 0.4 s.

The different analyses described above may be performed for walking, jogging, pacing, running, and/or jumping or other kinds of gait.

FIGS. 12a-c illustrate the general provisions on how to compute different parameters regarding the gait quality and/or gait-related health status of a human and how this information is used to improve performance and to detect increase in risk of injury and/or detect injuries and/or detect diseases.

In a first step 310, gait data are collected from the gait sensor devices 20a-b. In a further step 312, one or more gait parameters 210 are computed using the collected gait data 22a-b. As has already been described, the gait parameters 210 may also be computed by combining metadata 110 and gait data 22a-b. In one embodiment, although not illustrated, some of the gait parameters 210 can be assessed only using metadata 110.

In a next step 320, gait parameters 210 are compared against a normal baseline 321. If available, the gait parameters 210 may further be compared against gait data 22a-b history for the specific subject 323 or compare the gait parameters 210 against a baseline for the specific subject. If available, the parameters are also analyzed 320 by inputting expert knowledge 322.

Metadata 110 is/are collected in step 330. In a next step 340, metadata 110 are compared against a normal baseline 341. If available, the metadata 110 may further be compared against metadata history for the specific subject 343 or compare the metadata 110 against a baseline for the specific subject. If available, the data is also analyzed 340 by inputting expert knowledge 342.

The analyzed data from the gait parameters 210 and metadata 110 (as analysed in steps 320 and 340) are used to analyze 350 gait quality and/or gait-related health status of the subject. As described with relation to FIGS. 4a-b, this may be computed by a sensor controller 90, a web-based application programming interface 70, a storage unit 12, a computing unit 10, or by an external device 50. The analyzed health and performance data 350 is used to determine if one can see improvement(s) in health status 351 of the human 5, if there is no or at least one minor change 352 in the gait quality of the human 5 and/or if there is an increase in risk of one or more injuries and/or diseases 353 of the human 5. If the data indicates increase risk of injuries and/or diseases 353, the system may compute suggestions relating to changes 331 in the metadata 110 that would be beneficial. The analyzed gait quality and/or gait-related health status 350 and its findings is preferably transmitted 360 to a user. The information may be transmitted to an interface of the external device 50.

The findings 351, 352, 353 may be used to evaluate information on a short-term or long-term perspective. The findings 351, 352, 353 on the short-term 308 and/or long-term 309 perspective(s) may be used to assess the gait of the human 5, and/or to rank or evaluate the quality and effectiveness of some of the metadata 110. The analyzed data 350 could also be used to evaluate the effectiveness of the intervention and/or treatment, and/or evaluate the effectiveness of the medication itself or the dosage and/or the timing of the dosage. Metadata 110 that could be ranked is for example the accessory data 140, quality of service of a person, e.g. the person data 130, and/or the effectiveness of the training regime, e.g. the training data 150. It may for example be beneficial to rank an accessory data 140 in order to determine which type of equipment 146 that has the lowest or greatest impact on the gait of the human 5.

FIG. 12b illustrates how the normal baseline 321 is created using a database of many humans. In a first step, the gait data 22a-b of healthy subjects are collected 310, and the gait parameters 210 are computed 312 for the subjects. Moreover, metadata 110 is collected 332 from healthy humans. The information from both the computed gait parameters 210 and the collected metadata 110 is used to create 336 a normal baseline for all humans. The output from creating a normal baseline for all humans represents the normal health and performance status of a human. This evaluation is performed by the system 1 itself.

FIG. 12c illustrates how to set a baseline for a specific human. Gait data 22a-b is collected 310, and gait parameters 210 are computed 312. Moreover, metadata 110 is collected. The computed gait parameters 210 and the metadata 110 is used to set 334 a baseline for a specific human, whose output will then represent the normal gait-quality and/or gait-related health status of that specific human. The process described in FIG. 12c is preferably performed manually by an expert such as medical professionals, i.e. anyone from the person data 130.

In some embodiments, the computing unit 10 is configured to compute a total health score and/or risk of one or more injuries and/or diseases based on at least one metadata 110 and at least two gait parameters 210. The total score may be computed with no weight factor or may be computed using one or more weight factors. Weight factors are not needed if the different parameters/data are regarded as having the same importance, but may be beneficial if one or more of the gait analysis parameters are considered more important than others. In one embodiment the total score is a weighted average of at least two gait parameters 210 and one metadata 110. The total score may be used to either determine gait quality, gait-related health status compared to the subject 5 itself, and/or compared to the reference group data. The computing unit 10 may further be configured to rank the total scores of all analysed subjects 5 to generate a comprehensive list of the assessments.

The method of collecting and analysing the gait data 22a-b will now be described with reference to FIG. 13. The plurality of sensor devices 20a-b collect 310 sensor data/gait data 22a-b. For each sensor 20a-b, the system 1 collects the gait data 22a-b. From the gait data 22a-b two or more gait parameters 210 will be computed 312. In one embodiment, the gait parameters 210 comprises sets of acceleration signals, gyroscope signals and magnetometer signals.

The system then computes 370 if the data corresponds to a gait related activity or rest/inactive state by comparing 372 the data with predefined thresholds 374. If it is determined that the subject is in an active state, the system 1 computes 376, 378 an acceleration energy density spectrum as well as a gyroscope energy density spectrum using magnitude of the resultant acceleration and gyroscope signal obtained from each individual axes. If no active state is determined, the system 1 may collect additional gait data 22a-b and rerun the process according to FIG. 13. The computed gyroscope and acceleration energy density spectrums are valuable for analysing gait quality, i.e. to detect one or more injuries and/or diseases (at early stages) as they reveal any fluctuations in gait. This was discussed earlier with reference to FIG. 9. The energy density spectrums may be assessed to detect 380 abnormalities in gait quality of the human 5.

Gait abnormalities of the human may include hemiplegic gait, spastic gait, diplegic gait, neuropathic gait, myopathic gait, choreiform gait, ataxic gait, Duchenne gait, Parkinsonian gait (propulsive gait), and/or sensory gait. Any additional type of abnormal gait pattern known in the fields of medicine may be further be identified by assessing the energy density spectrums. Additionally, gait abnormalities may be associated with the musculotendinous unit, including abnormalities such as rhabdomyolysis, muscle contusion, myotendinous strain and tendon avulsion. Any of these abnormalities or other similar abnormalities may in some aspect affect the gait of the human 5. By for example analysing the energy density spectrum(s), cause, effect and possible remedies may be discovered. Accordingly, the energy density spectrum(s) is/are comprised in the stride characteristics being obtained and processed as gait data 22a-b.

Assessing and detecting 380 whether the subject's 5 gait quality and/or gait-related health status is/are related to any abnormalities involves either comparing the energy density spectrums from the acceleration and the gyroscope from each individual leg and/or by combining the energy density spectrums from the acceleration and the gyroscope to a combined energy density spectrum. The changes in gait speed and gait classification (type of gait) lead to changes in spectral energies in the individual acceleration and gyroscope energy density spectrums and the combined energy density spectrum of the two legs of the human 5. The system uses a moving window in time to track these changes in spectral energy to setup spectral-temporal boundaries. The maximum spectral-temporal energy peak within each boundary is identified as Heel strike and Toe off events. Once the gait event has been determined for one leg, all gait events from all legs are combined to create one single array of gait events. Expert knowledge about a specific gait may be used to identify gait sequences in gait events to further improve the classification of different gaits.

In addition, the gait data 22a-b collected 310 from sensor devices 20a-b may be combined with metadata 110 in order to compute 312 some of the gait parameters 210. In one example gait data 22a-b is combined with GPS data. In such embodiment, the GPS-signals are combined with the gait data 22a-b using sensor fusion techniques such as Kalman filtering to estimate speed, velocity and stride length.

More detailed flowcharts of how to compute gait parameters 210 are illustrated in FIGS. 14a-14e and the associated method steps 2000 to 2038.

FIG. 14a illustrates how to determine if a subject 5 is performing a gait-related activity or if it is inactive. The computing unit is further configured to receive 210 a set of acceleration signals from each sensor device 20a-b. For each set of received acceleration signals, a resultant acceleration signal is computed 2011. A moving std signal is then computed 2012 based on the resultant acceleration signal. A filtered acceleration signal is generated 2013 by performing 1-D morphological filtering of moving std. In a next step, if a percentage of values in the filtered acceleration signal is greater than a pre-determined activity threshold the method is continued to step 2015 where the procedure is repeated for all legs. If the step in 2014 is not fulfilled, it is determined that the subject 5 is inactive. In order to evaluate step 2014, an activity threshold may be used 2014b. In step 2016, if all legs fulfil the condition in step 2014, it is determined that the subject 5 is performing a gait-related activity. If not, it is determined that the subject is not performing a gait-related activity.

FIG. 14b illustrates how gait events are computed. The process in FIG. 10b is repeated for both legs individually. The process in FIG. 14b starts if it is determined that the subject 5 is performing a gait-related activity.

The system 1 receives the resultant acceleration signal 2017. In a next step, 2018, the wavelet transform is computed of the resultant acceleration signal. The acceleration energy density spectrum (aeds) is computed 2019 by summing the spectral energies at all scales in the wavelet transform (awt).

The system 1 receives 2020 a set of gyroscope signals from each sensor device 20a-b. A resultant gyroscope signal is computed 2021. A wavelet transform is computed 2022 of the gyroscope resultant signal. The gyroscope energy density spectrum (geds) is computed 2023 by summing the spectral energies at all scales in the wavelet transform (gwt). A combined energy density spectrum (ceds) is computed 2024 by taking the mean of the acceleration energy density spectrum (aeds) and the gyroscope energy density spectrum (geds). In step 2025, a running window in time is used to track the frequency/spectral changes over time in the combined energy density spectrum (ceds). The changes indicate the changes in gait frequency. In step 2026, the frequency tracking information is used to locate the regions of maximum spectral energy in the wavelet transform (awt) and (gwt). The maximum spectral-temporal energy peak within each region is identified as heel strike and toe off events.

FIG. 14c, illustrates gait classification. Based on the signals from the sensor devices 20a-b, the system uses time domain features 2028 and wavelet domain features 2029 to classify gait 2030, for instance whether the subject is walking, running, jumping or striding. The time domain features may be all moments of the acceleration signals and gyroscope signals, such as mean values, median, variance and kurtosis. The time domain feature may also include filtering the acceleration signal. The wavelet domain features may be extracted from the energy density spectrums, such as for example inflection points, area under the energy density spectrums, as well as moments of the energy density spectrum signals.

FIG. 14d illustrates segmentation within each gait type. In step 2031, a filtered acceleration signal (gf) is generated by performing 1-D morphological filtering of resultant acceleration signal (ar). In step 2032, a convoluted signal (cs) is computed by convoluting (gf) with first order derivative of a gaussian function. All inflection points in the convoluted signal is located 2033. In step 2034, the time-location of the inflection points whose magnitude is above a pre-determined threshold gives the gait transitions, i.e. segments of gait intensities within each gait type.

In FIG. 14e, all gait events from both legs of the subject 5 are combined 2035 to create one single array of gait events. Expert knowledge about a specific gait may be used 2036 to identify gait sequences in (ge) from the classified gait segments (gs). In step 2037, the initial signal (ar) and (gr) are classified into gait segments based on different gait types and intensities within each gait type. For each segment, gait parameters are computed 2038.

In one embodiment, the computing unit 10 is further configured to compute statistical data of at least one of the gait parameters 210 and/or metadata 110. Statistical data may be used to more accurately assess future health and performance assessment of or previously encountered subjects 5. In this regard, the computing unit 10 further comprises self-learning features. For instance, the system may perform autonomous classifications based on previously analysed gait patterns. The training dataset used by the computing unit 10 preferably comprises the reference group data and/or individual previously generated assessments of the specific gait analysis subject 5. The classifications may relate to one or more of the disorders/diseases/injuries/etc. as discussed herein, and the classifications are preferably made based on the one or more metadata 110 and/or one or more gait parameters 210. To perform the classifications and thus more accurately determine a gait quality, the computing unit 10 may implement binary, multi-class, or multi-label classification and/or clustering algorithms. For instance, algorithms such as logistic regression, support vector machines, kernel estimation, decision trees and/or artificial neural networks may be utilized. Upon accurately or inaccurately having determined a gait quality, the learning parameters are used for subsequent training of the algorithm to improve its accuracy.

For the computed data discussed above, the storage unit 12 may be configured to store the statistical data, the gait pattern indices and the health and performance assessment. Further, the storage unit 12 may further be configured to transmit this data to the external device 50.

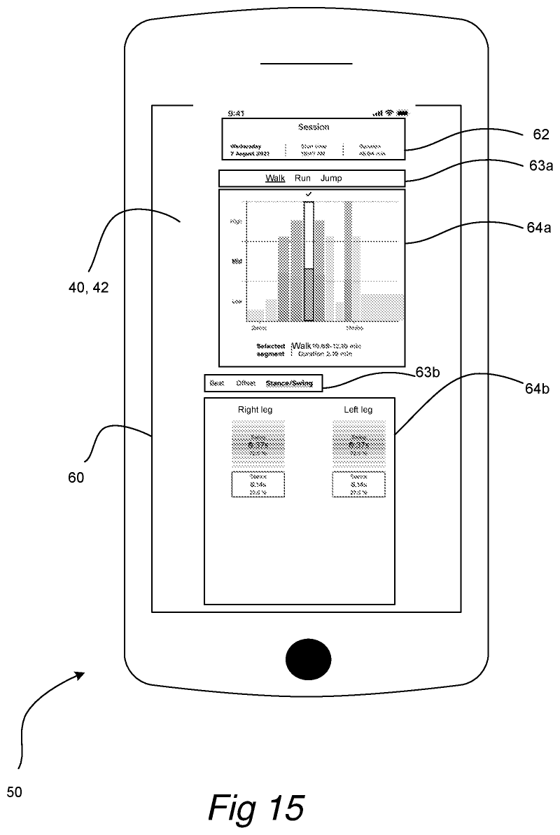

Upon having received any of the data transmitted by the storage unit 12, the external device 50 is configured to present information to the user of the external device 50 on the display 60. This is illustrated in FIG. 15. The external device 50 is configured to present one or more of metadata 110, gait parameters 210 and/or the final assessment regarding gait quality and/or gait-related health and performance.

The presentation of information is preferably done using any comprehensive graphical user interface being directly intractable via the display 60 by the user of the external device 50. The information may be retrieved as a request from the external device 50 to the storage unit 12. The information may also be transmitted in real-time.

In an embodiment of the invention, the computing unit 10 is further configured to generate and transmit a deviating signal indicating that something or some data in the graphs/parameters/data is abnormal. The deviating signal may be generated as a result of a detected value greatly diverging from an expected value relating any of the parameters of the assessment. For instance, if an essential classification which requires immediate attention has been made, this may be transmitted to the external device 50. Consequently, the external device 50 is configured to present said received deviating signal to the user. Furthermore, the deviating signal may also be broadcasted to many devices if necessary. A deviating report of the cause of the deviating signal may also be generated and transmitted to the external device 50. The external feedback may be in the form of a sound, vibrations, text message, e-mail, phone call, etc.

In FIG. 15, an illustration of an external device 50 is shown. More specifically, the display 60 of an external device 50 is depicted. The display 60 may be configured to present any type of information being produced by, associated with or in some sense related to the system 1. Accordingly, performance and/or health of different entities (subjects, medical professional etc.) may be viewed in the display 60.

The display 60 preferably comprises a graphical user interface (GUI), such as the one shown in FIG. 15. The GUI may comprise an upper tab 62 comprising general information of what type of information is being currently presented on the display 60. In the shown example, the upper tab 62 describes that the presented information is related to a particular training session, i.e. that the GUI comprises information related to the training data 150. Moreover, a particular date, start time and duration is shown. The upper tab 62 may alternatively describe that the GUI presents other types of metadata 110 or gait parameters 210, such as subject data 120, person data 130, accessory data 140, stride details, or activity details 230.

The GUI may further comprise a menu tab 63a-b wherein the user of the device 50 may switch between specific information related to the current e.g. training session. FIG. 15 currently shows that the user has selected to view data related to walk in a first information box 64a and data related to Stance/Swing in a second information box 64b. The GUI may comprise any number of simultaneously active information boxes 64a-b such that the user may customize its appearance based on interest.

The information presented in the GUI of the display 60 of the external device 50 can for example show information related to different training routines; the subject's 5 movement over slopes; the subject's 5 movement clockwise around a lap; the subject's 5 movement anti-clockwise around a lap; and so forth. Accordingly, the display 60 may indicate how the subject 5 is acting when walking in a straight line, or running in lunges in clockwise or anti-clockwise direction, respectively. The information presented in the GUI may be viewed for any number of subjects 5 simultaneously (e.g. in different information boxes 64a-b) or one by one.

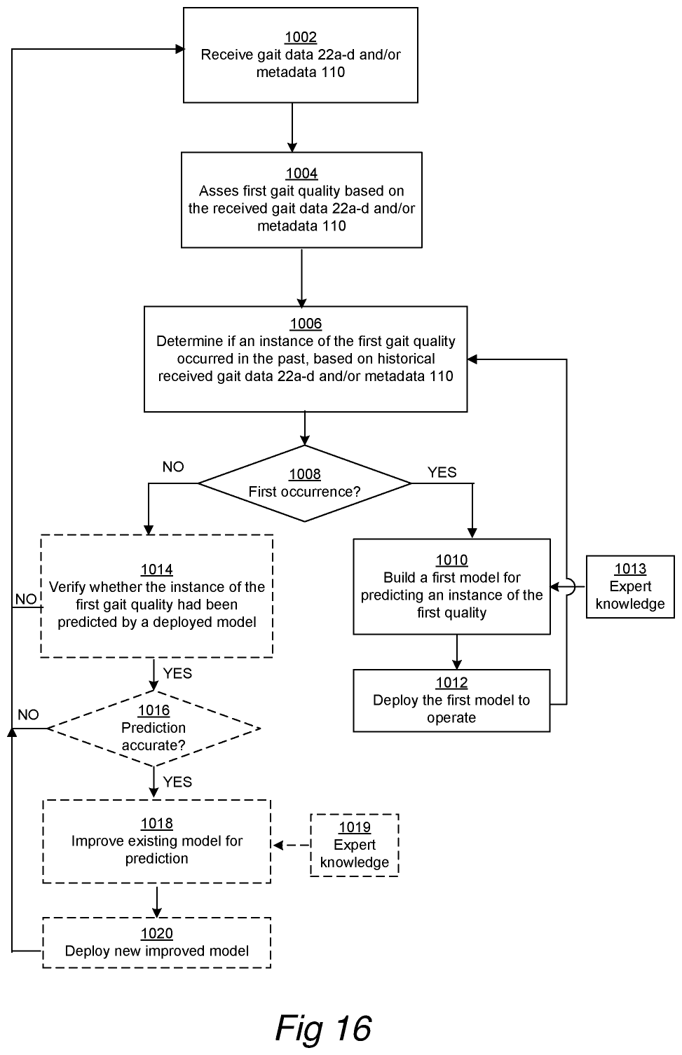

One embodiment of a method of predicting human gait quality of a human is illustrated in FIG. 16. The method preferably comprises receiving, 1002, gait data 22a, and/or metadata 110 as has previously been described. The method further comprises assessing, 1004, a first human gait quality based on the received gait data 22a-b and/or metadata 110; and then determining, 1006, 1008 if an instance of the first human gait quality occurred in the past, based on historically received gait data 22a-b and/or metadata 110.

If this is a first occurrence of the first human gait quality, 1008-yes, then based on gait data 22a-b and/or metadata 110 received before detecting the first human gait quality and building a first model for predicting an instance of the first human gait quality. Once the first model is built, the first model is deployed to operate, 1012. If this is not a first occurrence of said first human gait quality, 1008-no, the method comprises verifying, 1014, whether this instance of the first human gait quality had been predicted by a deployed model for predicting an instance of the first human gait quality. If the 10 instance of the first human gait quality had not been predicted by the deployed model predicting an instance of the first human gait quality, or the prediction was not accurate, step 1016-no, the method comprises developing another model for predicting an instance of the first human gait quality and deploying another model to operate. In a preferred embodiment, the operation of developing another model for predicting an instance of the first human gait quality may comprise re-training the first model on a new set of gait data 22a-b and/or metadata 110.

Preferably, the method may further comprise determining if in the received gait data 22a-b and/or metadata 110 one or more human gait quality coincide with the first human gait quality and then use the gait data 22a-b and/or metadata 110 being indicative of the one or more human gait quality coinciding with the first human gait quality to build the first model for predicting an instance of the first human gait quality. Hereby, additional influencing factors (apart from the gait data 22a-b and/or metadata 110 used to detect the human gait qualities) are used to develop (build) the prediction model to improve its accuracy of prediction.

In yet another alternative embodiment the method according to embodiment the method comprises clustering at least some of the received time series of the gait data 22a-b and/or metadata 110; into at least one cluster and then using the time series of gait data 22a-b and/or metadata 110; from the at least one cluster for building the first model for predicting an instance of said first human gait quality.

This embodiment further improves accuracy of the prediction model because it exploits relationships between the gait data 22a-b and/or metadata 110 that led to detection of the human gait quality and other time series of the gait data 22a-b and/or metadata 110. The relationships between the time series in a cluster are not only temporal but may also be of a different nature (e.g. based on temperature at the location where the human is located or physical location, etc.). Thus it is possible to detecting trends in at least some of the time series of gait data 22a-b and/or metadata 110 that are indeed related with the first human gait quality but occur prior to the first human gait quality. This, in turn, allows for more accurate prediction of human gait qualities. In a further preferred embodiment, the received gait data 22a-b and/or metadata 110 comprise gait data 22a-b and/or metadata 110 received as individual values and the method comprises converting the individual values to time series of values. In one exemplary embodiment the computing unit 10 is further configured to build a model using the received gait data 22a-b and/or metadata 110 and deploying the model for predicting of human gait quality. In one exemplary embodiment the computing unit 10 is configured to receive gait data 22a-b and/or metadata 110 received as time series of values representing gait characteristics and/or metadata 110 associated with the human.

The computing unit 10, is also operative to detect a first human gait quality for the human and determine if an instance of the first human gait quality occurred in the past based on historical gait data 22a-b and/or metadata 110. If this is a first occurrence of the first human gait quality, then based on gait data 22a-b and/or metadata 110 received before detecting the first human gait quality, the computing unit 10, is operative to build a first model for predicting an instance of said first human gait quality and then deploy the first model in the to operate.

In a preferred embodiment to develop another model for predicting an instance of the first human gait quality the computing unit 10 is operative to re-train the first model on a new set of gait data 22a-b and/or metadata 110. In yet another preferred embodiment to develop another model for predicting an instance of the first human gait quality the apparatus is operative to update the first model.

Preferably, the computing unit 10, is further operative to determine if in the received gait data 22a-b and/or metadata 110 one or more human gait qualities coincide with the first human gait quality and use the received gait data 22a-b and/or metadata 110 indicative of the one or more human gait qualities coinciding with the first human gait quality for building the first model for predicting an instance of the first human gait quality.

Preferably, the computing unit 10, is further operative to cluster at least some of the received gait data 22a-b and/or metadata 110 into at least one cluster and use the time series of the gait data 22a-b and/or metadata 110 from the at least one cluster for building the first model for predicting an instance of the first human gait quality.

In a preferred embodiment the received gait data 22a-b and/or metadata 110 data received as individual values and the computing unit 10, is operative to convert the individual values to time series of values.

The advantages of the present solution include (but are not limited to) the following: Human gait qualities are predicted before they occur, and remedial measures are taken to avoid human gait qualities that can be harmful to the human. This enables a proactive approach of autonomous human gait quality management compared to the current reactive approach. Data e.g. gait data 22a-b and/or metadata 110 are autonomously determined for the incident/anomaly rather than purely relying on historical knowledge base and/or medical gait expertise. Autonomous recommendation becomes possible due to discovery of determining impacting factors of human gait quality. When the impacting factors are known then recommending solutions is feasible and can be derived from knowledge of how to impacting factors influence the human gait quality.

The present disclosure provides a solution for human gait quality prediction using a model developed by a machine learning algorithm in which the machine learning algorithm uses historical gait data 22a-b and/or metadata 110 for training. Once the model is ready, it is deployed and operates on incoming gait data 22a-b and/or metadata 110.

Accuracy of prediction of human gait quality by the model is verified in order to improve the model and achieve higher accuracy of prediction. The amount of historical gait data 22a-b and/or metadata 110 increase as the data is collected, so if prediction is not accurate enough (e.g. gets less accurate than in previously) the machine learning algorithm re-trains on new (and in some embodiments bigger set of data) to develop an improved model for human gait quality. If a new human gait quality is detected (i.e. a new type of human gait quality) the machine learning algorithm develops a model in run time for predicting instances of this newly observed human gait quality. In a preferred embodiment there are different models deployed for predicting different types of human gait quality (e.g. incidents related to health status of the human).

Using the initial human gait quality that led to detection of an human gait quality and any additional human gait parameters and/or trends a new machine learning prediction model is built at runtime and deployed to predict future occurrence human gait parameters. The new machine learning prediction model preferably may also be evaluated before being deployed. The evaluation may be carried out by running the model on gait data 22a-b and/or metadata 110 which, preferably, is also a set of historical gait data 22a-b and/or metadata 110 that exhibits the incident for detection of which the model has been developed, whereas the gait data 22a-b and/or metadata 110 was not used for development of the prediction model.

Also preferably, further evaluation of the prediction model is carried out in run time—the model predicts an human gait quality and the prediction is then verified against receive gait data 22a-b and/or metadata 110. If the accuracy of the prediction is not as good as expected a new prediction model may be developed. In addition to correlation of human gait parameters or trends in gait parameters to build the prediction model a cluster of time series of human gait parameters may be used as a possible factor for prediction.

The on-demand created model can predict future human gait quality based on historical gait data 22a-b and/or metadata 110 that can potentially help in mitigating human gait quality before an human gait quality problem occurs again.

The invention has been described above in detail with reference to embodiments thereof. However, as is readily understood by those skilled in the art, other embodiments are equally possible within the scope of the present invention, as defined by the appended claims.

1. An analysis system for assessing gait quality and/or gait-related health status of a human, the analysis system comprising:
a first sensor device arranged at a region of a first leg of the human;
a second sensor device arranged at a region of a second leg of the human;
wherein the first and the second sensor devices each comprises at least one 3-axis accelerometer and at least one 3-axis gyroscope;
wherein the first and the second sensor devices are configured to provide gait data; and
a computing unit configured to
receive the gait data from the first and the second sensor devices,
analyze the received gait data for determining at least one gait parameter related to stride characteristics of the human, wherein the at least one gait parameter comprises information of at least one computed energy density spectrum, and
analyze the at least one gait parameter to assess gait quality and/or gait-related health status of the human.

2. The analysis system according to claim 1, wherein the at least one computed energy density spectrum comprises at least one energy density spectrum computed from both or either one of sets of acceleration signals or gyroscope signals included in the gait data.

3. The gait analysis system according to claim 2, wherein the computing unit is further configured to analyze the at least one energy density spectrum by:
measuring a variability by comparing each energy density spectrum to itself over a predetermined time period, and/or
measuring a symmetry by comparing an energy density spectrum of a left leg of the human to an energy density spectrum of a right leg of the human, and/or
measuring a normality by comparing each energy density spectrum to at least one energy density spectrum of a leg from a reference population group exhibiting no gait pathology.

4. The analysis system according to claim 2, wherein the computing unit is configured to compute accelerometer energy density spectrums by:
receiving the sets of acceleration signals;
for each set of received acceleration signals, computing a resultant acceleration signal;
based on the computed resultant acceleration signals, determining if the human is performing a gait related activity or is inactive; and
if it is determined that the human is performing a gait related activity, computing an accelerometer energy density spectrum for each resultant acceleration signal, wherein each accelerometer energy density spectrum corresponds to one leg of the human.

5. The gait analysis system according to claim 4, wherein determining if the human is performing a gait related activity further involves:
computing a moving standard deviation signaler of the resultant acceleration signals;
generating a filtered acceleration signal by performing filtering of the computed moving standard deviation signal; and
determining if a total number of elements of the filtered acceleration signal having a value greater than or equal to a value of a corresponding element of a predetermined walking threshold.

6. The gait analysis system according to claim 2, wherein the computing unit is configured to compute gyroscope energy density spectrums by:
receiving the sets of gyroscope signals;
for each set of received gyroscope signals, computing a resultant gyroscope signal; and
for each resultant gyroscope signal, computing a gyroscope energy density spectrum, wherein each gyroscope energy density spectrum corresponds to one leg of the human.

7. The gait analysis system according to claim 4, wherein the computing unit is configured to combine the accelerometer energy density spectrum and a gyroscope energy density spectrum for assessing gait quality and/or gait-related health status of the human.

8. The gait analysis system according to claim 4, wherein the accelerometer energy density spectrum and/or a gyroscope energy density spectrum is used to measure fluctuations in gait over time.

9. The analysis system according to claim 1, wherein the computing unit is further configured to:
receive at least one metadata associated with the human,
analyze the at least one gait parameter and the at least one metadata to assess gait quality and/or gait-related health status of the human.

10. The analysis system according to claim 9, wherein the at least one metadata comprises one or more of:
information of subject data of the human,
information of person data of persons connected to the human,
information of accessory data related to accessories of the human, and
information of training data of the human.

11. The analysis system according to claim 9, wherein the at least one metadata is based on data received from at least one additional sensor and/or based on data being inputted to the system by a user.

12. The analysis system according to claim 11, wherein the at least one additional sensor is one or more of: a GPS-sensor, a temperature sensor, a weather sensor, and a pulse sensor.

13. The analysis system according to claim 9, wherein the at least one gait parameter and the at least one metadata are analyzed by comparing them to one or more baselines and/or to historical data.

14. The gait analysis system according to claim 9, wherein the computing unit is further configured to compute statistical data and/or historical data of the at least one metadata and the at least one gait parameter.

15. The analysis system according to claim 9, wherein the computing unit is further configured to store the assessed gait quality and/or gait-related health status and/or to communicate the gait quality and/or gait-related health status to an external device having a display, wherein the external device is configured to present the assessed gait quality and/or gait-related health status to a user.

16. The gait analysis system according to claim 15, wherein the computing unit is further configured to generate and transmit a deviating signal to the external device if the at least one metadata and/or the at least one gait parameter exceeds a predetermined deviating threshold value.

17. The analysis system according to claim 1, wherein the computing unit is configured to analyze the received gait data for determining at least two gait parameters, wherein the second one gait parameter comprises one or more of:
information relating to stride details of the human,
information relating to activity details of a training session of the human, and
information relating to gait of the human.

18. The analysis system according to claim 1, wherein the assessed gait quality and/or gait-related health status is used to detect at least one of:
one or more improvements in health status of the human,
no or at least one change in the gait quality of the human, and/or
an increase in risk of one or more injuries and/or diseases of the human.

19. A method for assessing gait quality and/or gait-related health status of a human being equipped with a first sensor device at a region of a first leg of the human and a second sensor device at a region of a second leg of the human, wherein the first and the second sensor devices each comprises at least one 3-axis accelerometer and at least one 3-axis gyroscope, and wherein the first and the second sensor devices are configured to provide gait data, the method comprising:
receiving the gait data from the first and the second sensor devices;
analyzing the received gait data for determining at least one gait parameter related to stride characteristics of the human, wherein the at least one gait parameter comprises information of at least one computed energy density spectrum; and
analyzing the at least one gait parameter to assess gait quality and/or gait-related health status of the human.
